(19) United States
(12) Patent Application Publication
(10) Pub. No.: US 2005/0002994 A1
(43) Pub. Date: Jan. 6, 2005

(54) Cosmetic or dermatological impregnated tissues

(75) Inventors: Anja Goppel, Hamburg (DE); Andreas Bleckmann, Ahrensburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 10/839,256

(22) Filed: May 6, 2004

Related U.S. Application Data
(63) Continuation of application No. PCT/EP02/11995, filed on Oct. 26, 2002.

(30) Foreign Application Priority Data
Nov. 7, 2001 (DE) 101 54 627.0

(51) Int. Cl. A61K 9/70

(52) U.S. Cl. 424/443; 442/123

(57) ABSTRACT

A cosmetic or dermatological tissue comprising a water-insoluble nonwoven impregnated and/or moistened with a cosmetic or dermatological W/O emulsion comprising an emulsifier system of an O/W emulsifier having an HLB value of >10 and a silicone emulsifier (W/S) having an HLB value of ≦8 and/or a W/O emulsifier having an HLB value of <7.

### CROSS-REFERENCE TO RELATED APPLICATIONS

The present application is a continuation of International Application No. PCT/EP02/11995 filed Oct. 26, 2002, the entire disclosure whereof is expressly incorporated by reference herein, which claims priority under 35 U.S.C. § 119 of German Patent Application No. 101 54 627.0, filed Nov. 7, 2001.

### BACKGROUND OF THE INVENTION

1. Field of the Invention

The present invention relates to cosmetic and dermatological tissues which are moistened with highly liquid cosmetic and dermatological impregnation solutions—in particular with highly liquid cosmetic and dermatological water-in-oil emulsions (W/O emulsions) which are long-term stable. In particular, the invention relates to cosmetic and dermatological impregnated, optionally surface-structured, care, cleansing and deodorant tissues, and impregnated tissues for the control and prevention of skin diseases (such as acne, sunburn etc.) and those which specifically care for the skin after sunbathing and decrease the after-reactions of the skin to the action of UV radiation. The present invention further relates to impregnation solutions which are suitable for the impregnation of tissues of this type.

2. Discussion of Background Information

Impregnated tissues are widely used in all sorts of areas as articles of everyday necessity. Inter alia, they allow efficient and skin-caring cleansing and care, particularly also in the absence of (running) water. Here, the actual article of daily use consists of two components:
a) a dry tissue, which is constructed from the materials such as paper and/or all sorts of mixtures of natural or synthetic fibers, and
b) a low-viscosity impregnation solution.

Cosmetic or dermatological tissues can consist either of water-soluble (e.g. such as toilet paper) or of water-insoluble materials. The tissues can further be smooth or alternatively surface-structured. Surface-structured tissues are produced, for example, based on cellulose and are used in particular as household tissues and for perianal cleansing. Their structure is produced by mechanical embossing by means of calendering rolls. Tissues of this type have a low resistance to tearing with at the same time great roughness and hardness. They are therefore only limitedly suitable for use on the human skin.

Conventional impregnation solutions for water-insoluble nonwoven materials have hitherto had the deficiency of low long-term stability. Emulsions of this type are prone, in particular at high environmental temperature, to phase separation, which is a crucial disadvantage for the impregnation process and also for the final quality of the end product.

The long-term stability of known impregnation solutions is in general guaranteed by the use of increased emulsifier concentrations and also high energy input—for example on repeated homogenization.

It would be desirable to have available impregnation solutions which are stable long-term for application to water-insoluble nonwoven materials, which do not exhibit the disadvantages of the prior art and which are highly liquid emulsions which are stable long-term even at low emulsifier contents, which have to be homogenized only slightly and can contain more caring lipids and water-insoluble active ingredients.

### SUMMARY OF THE INVENTION

The present invention provides a cosmetic or dermatological tissue which comprises a water-insoluble nonwoven which is impregnated and/or moistened with a cosmetic or dermatological W/O emulsion. This emulsion comprises (a) a water phase, (b) at least one oil phase which comprises one or more oils and/or one or more lipids and (c) an emulsifier system of
(A) at least one O/W emulsifier having an HLB value of >10;
(B) at least one silicone emulsifier (W/S) having an HLB value of ≦8, and/or
(C) at least one W/O emulsifier having an HLB value of <7.
   The emulsion has a viscosity of less than 2,000 mPa·s and a silicone oil content of not more 25% by weight.

In one aspect of the tissue, the weight ratio of the nonwoven and the W/O emulsion may be from 5:1 to 1:5.

In another aspect, the nonwoven may comprise a structured nonwoven.

In yet another aspect, the nonwoven may comprise an unstructured nonwoven.

In a still further aspect of the tissue, the nonwoven may comprise a jet consolidated nonwoven and/or a water jet-embossed nonwoven.

In another aspect, the nonwoven may have a thickness of from 0.4 mm to 1.5 mm and/or an area weight of from 35 to 120 g/m². For example, the nonwoven may have a thickness of from 0.6 mm to 0.9 mm and an area weight of from 40 to 60 g/m².

In another aspect, the nonwoven may comprise fibers of a mixture of 70% by weight of viscose and 30% by weight of polyethylene terephthalate.

In yet another aspect, the nonwoven may comprise fibers which have a water absorption rate of more than 60 mm/10 min and/or a water absorption capacity of more than 5 g/g, e.g., a water absorption rate of more than 80 mm/10 min and/or a water absorption capacity of more than 8 g/g.

In another aspect of the present invention, the at least one silicone emulsifier B may comprise an alkylmethicone copolyol and/or an alkyl dimethicone copolyol. For example, the at least one silicone emulsifier B may comprise an emulsifier of the formula 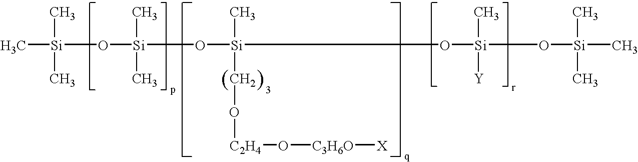
in which X and Y independently represent H, a branched or unbranched alkyl group, an acyl group and an alkoxy group having 1-24 carbon atoms, p is a number of from 0-200, q is a number of from 1-40, and r is a number of from 1-100.

In a still further aspect of the issue of the present invention, the at least one W/O emulsifier C may comprise at least one polyglycerol emulsifier.

In another aspect, the at least one O/W emulsifier A may comprise at an ethoxylated polysorbate and/or an ethoxylated stearate and/or a phosphate emulsifier and/or a sulfate emulsifier.

In another aspect, the emulsion may comprise a total concentration of A, B and C of from 0.1% to 15% by weight, e.g., of from 0.5% to 10% by weight, or of from 2% to 10% by weight.

In yet another aspect, the weight ratio A:B:C is expressed as a:b:c and a, b and c may be rational numbers of from 1 to 5, preferably of from 1 to 3.

In another aspect, the emulsion may comprise from 0.5% to 5.0% by weight of the at least one silicone emulsifier B.

In another aspect of the tissue of the present invention, the emulsion may comprise at least 2% by weight of one or more silicone oils which comprise a cyclic silicone and/or a linear silicone and/or a derivative thereof.

In yet another aspect, the emulsion may comprise from 2% to 25% by weight of at least one silicone oil, e.g., from 5% to 20% by weight, or from 10% to 20% by weight of at least one silicone oil.

In a still further aspect, the at least one oil phase may comprise a polar oil and/or a carboxylic acid ester, and/or a dialkyl ether and/or a dialkyl carbonate. For example, the at least one oil phase may comprise a C₁₂₋₁₅ alkyl benzoate.

In another aspect, the emulsion may comprise from 1% to 90% by weight of the at least one oil phase, e.g., from 2.5% to 80% by weight, or from 5% to 70% by weight of the at least one oil phase.

In another aspect, the emulsion may further comprise at least one light protection filter which is selected from oil-soluble and water-soluble light protection filters. The at least one light protection filter may comprise one or more UV filters, e.g., a triazine, a sulfonated UV filter, a UV filter which is liquid at room temperature, an inorganic pigment and/or a benzotriazole. Preferably, the one or more UV filters may comprise at least one of 2,4-bis{[4-(2-ethylhexyloxy)2-hydroxy]phenyl}-6-(4-methoxyphenyl)-1,3,5-triazine, dioctylbutylamidotriazine, 4,4′,4″-(1,3,5-triazine-2,4,6-triyltriimino)trisbenzoic acid tris(2-ethylhexyl ester), phenylene-1,4-bis(2-benzimidazyl)-3,3′,5,5′-tetrasulfonic acid bis sodium salt, 2-phenylbenzimidazole-5-sulfonic acid, terephthalidene dicamphorsulfonic acid, 4-methoxycinnamic acid (2-ethylhexyl)ester, 2-ethylhexyl-2-cyano-3,3-diphenyl acrylate, 2-ethylhexyl 2-hydroxy-benzoate, homomenthyl salicylate, TiO₂, ZnO, 2,2′-methylenebis(6-(2H-benzotriazol-2-yl)-4-( 1,1,3,3-tetramethylbutyl)phenol, and 2-(2H-benzotriazol-2-yl)-4-methyl-6-[2-methyl-3-[1,3,3,3-tetramethyl[(trimethylsilyl)oxy]disiloxanyl]propyl]-phenol.

In another aspect, the emulsion may further comprise an additive and an active ingredient. For example, the emulsion may further comprise a repellent, a self-tanning agent and/or a pigment.

In another aspect, the emulsion may further comprise vitamin E and/or a derivative thereof and/or α-glycosylrutin and/or a derivative thereof.

In yet another aspect, the emulsion may further comprise at least one component selected from moisturizers, waxes, surfactants, preservatives, antioxidants, dyes, plant extracts, deodorants, antiperspirants, dermatologically active ingredients, and perfumes.

In a still further aspect, the emulsion may have a high water resistance.

The present invention also provides various products which comprise the tissue of the present invention, e.g., a skin care product, an insect repellent, a self-tanning product, a sunscreen product, a product for the treatment or prophylaxis of light-related skin ageing, a skin moisturizing product, a baby care product and a skin cleansing product.

The present invention also provides the above O/W emulsion for use in the production of the tissue of the present invention, including the various aspects thereof.

The present invention further provides a process for manufacturing the tissue of the present invention. This process comprises providing a water-insoluble nonwoven and impregnating and/or moistening the nonwoven with the W/O emulsion of the present invention.

It is surprising that cosmetic and dermatological tissues comprising a water-insoluble nonwoven which is impregnated or moistened with cosmetic and dermatological W/O impregnation emulsions, which in addition to further cosmetic/dermatological additives or excipients has an emulsifier system of
A at least one O/W emulsifier having an HLB of >10,
B at least one silicone emulsifier (W/S) having an HLB of ≦8 and/or
C at least one W/O emulsifier having an HLB of <7
   and a viscosity of less than 2000 mPa.s, a silicone oil content of not more than 25% by weight (based on the total weight of the preparation) and one or more oil phases comprising lipids and/or oils, may remedy the disadvantages of the prior art.

The tissues according to the invention represent the combination of a soft, water-insoluble, nonwoven material with highly liquid cosmetic and dermatological W/O impregnation emulsions. They are extremely satisfactory in every respect and are accordingly very particularly suitable for use as a basis for preparation forms having a variety of application purposes. The tissues according to the invention exhibit very good sensory and cosmetic properties and are further distinguished by outstanding skin care data.

The nonwoven material is preferably consolidated by jets of water in the production process as a spunlace material. The tissues according to the invention can be either structured or unstructured (“smooth”). If the material is to be the structured, the structuring is advantageously likewise carried out by means of jets of water. By means of this structuring, for example, a uniform sequence of elevations and depressions results in the material.

In combination with suitable impregnation solutions, this structuring by means of its elevations makes possible both a better access to hollows in the human skin and by means of its structural valleys to an increased dirt absorption capacity. This leads overall to a markedly improved cleansing power.

A better access to depressions in the human skin is moreover of particular importance for the control of skin diseases and skin irritations, and for the effective display of a deodorant action.

Depending on the tissue employed, the weight ratio of the unimpregnated tissue to the W/O emulsion is may be in the range of from 5:1 to 1:5. By means of this, drip-free application of the impregnated tissue is guaranteed.

In particular, structured cosmetic or dermatological tissues are therefore preferred according to the invention.

The cosmetic and dermatological W/O impregnation emulsions with which the tissues according to the invention are moistened can be present in various forms.

They are preferably highly liquid to sprayable and have, for example, a viscosity of less than 2000 mPa·s, in particular of less than 1500 mPa·s (measuring apparatus: Haake Viskotester VT-02 at 25° C.).

The preparations according to the invention are extremely satisfactory preparations in every respect. In particular, it was surprising that the emulsions produced from the preparations according to the invention have a high solubility for UV filters from the group of the triazines and thus the achievement of a high UVA & UVB protection factor is possible. In addition, repellents and also self-tanning substances (e.g. dihydroxyacetone) can be stably incorporated in these novel W/O emulsions.

Accordingly, preparations within the meaning of the present invention are very particularly suitable for use as a basis for product forms having a variety of application purposes.

Impregnation emulsions according to the invention can also contain only one of the emulsifiers B and C—depending on the content of silicone oils and lipids—in addition to the O/W emulsifier A.

According to the invention, the silicone emulsifiers B can advantageously be selected from the group of the alkylmethicone copolyols and/or alkyldimethicone copolyols, in particular from the group of compounds which are characterized by the following chemical structure: 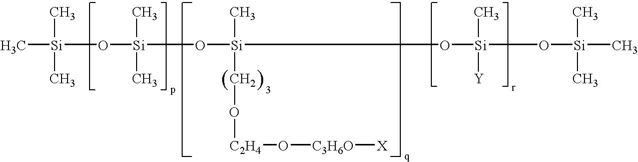
in which X and Y are chosen independently of one another from the group consisting of H (hydrogen), and the branched and unbranched alkyl groups, acyl groups and alkoxy groups having 1-24 carbon atoms, p is a number from 0-200, q is a number from 1-40, and r is a number from 1-100.

An example of silicone emulsifiers to be used particularly advantageously within the meaning of the present invention are dimethicone copolyols which are marketed by the company Th. Goldschmidt AG under the trade names ABIL® B 8842, ABIL® B 8843, ABIL® B 8847, ABIL® B 8851, ABIL® B 8852, ABIL® B 8863, ABIL® B 8873 and ABIL® B 88183.

A further example of interface-active substances to be used particularly advantageously within the meaning of the present invention is cetyl dimethicone copolyol which is marketed by the company Goldschmidt AG under the trade name ABIL® EM 90.

A further example of interface-active substances to be used particularly advantageously within the meaning of the present invention is dimethicone copolyol cyclomethicone which is marketed by the company Goldschmidt AG under the trade name ABIL® EM 97.

Furthermore, the emulsifier laurylmethicone copolyol which is obtainable under the trade name Dow Corning® 5200 Formulation Aid from the company Dow Corning Ltd. has turned out to be very particularly advantageous.

A further advantageous silicone emulsifier is ‘Octyl Dimethicone Ethoxy Glucoside’ from Wacker.

The total amount of silicone emulsifiers B used according to the invention in the cosmetic or dermatological preparations according to the invention is advantageously in the range of from 0.1-10.0% by weight, preferably 0.5-5.0% by weight, based on the total weight of the preparations.

According to the invention, the W/O emulsifier(s) C are preferably chosen from the following group: sorbitan stearate, sorbitan oleate, lecithin, glyceryl lanolate, lanolin, microcrystalline wax (Cera microcristallina) as a mixture with paraffin oil (liquid paraffin), ozocerite, hydrogenated castor oil, glyceryl isostearate, polyglyceryl 3-oleate, wool wax acid mixtures, wool wax alcohol mixtures, pentaerithrityl isostearate, polyglyceryl 3-diiso-stearate, sorbitan oleate as a mixture with hydrogenated castor oil, beeswax (Cera alba) and stearic acid, sodium dihydroxycetyl phosphate as a mixture with isopropyl hydroxycetyl ether, methyl glucose dioleate, methyl diglucose dioleate as a mixture with hydroxystearate and beeswax, mineral oil as a mixture with petrolatum and ozocerite and glyceryl oleate and lanolin alcohol, petrolatum as a mixture with ozocerite and hydrogenated castor oil and glyceryl isostearate and polyglyceryl 3-oleate, PEG-7 hydrogenated castor oil, sorbitan oleate as a mixture with PEG-2 hydrogenated castor oil, ozocerite and hydrogenated castor oil, sorbitan isostearate as a mixture with PEG-2 hydrogenated castor oil, polyglyceryl 4-isostearate, polyglyceryl 4-isostearate, hexyl laurate, acrylate/C₁₀₋₃₀-alkyl acrylate crosspolymer, sorbitan isostearate, poloxamer 101, polyglyceryl 2-dipolyhydroxystearate, polyglyceryl 3-diisostearate, polyglyceryl 4-dipolyhydroxystearate, PEG-30 dipolyhydroxystearate, diisostearoyl polyglyceryl 3-diisostearate, polyglyceryl 2-dipolyhydroxystearate, polyglyceryl 3-dipolyhydroxystearate, polyglyceryl 4-dipolyhydroxystearate, polyglyceryl 3-dioleate.

According to the invention, the O/W emulsifier(s) A are preferably chosen from the following group: Glyceryl stearate as a mixture with ceteareth-20, ceteareth-25, ceteareth-6 as a mixture with stearyl alcohol, cetyl stearyl alcohol as a mixture with PEG-40 castor oil and sodium cetyl stearyl sulfate, triceteareth 4-phosphate, glyceryl stearate, sodium cetyl stearyl sulfate, lecithin trilaureth-4 phosphate, laureth-4 phosphate, stearic acid, propylene glycol stearate SE, PEG-25 hydrogenated castor oil, PEG-54 hydrogenated castor oil, PEG-6 caprylic acid/capric acid glycerides, glyceryl oleate as a mixture with propylene glycol, PEG-9 stearate, PEG-20 stearate, PEG-30 stearate, PEG-40 stearate, PEG-100 stearate, ceteth-2, ceteth-20, polysorbate-20, polysorbate-60, polysorbate-65, polysorbate-100, glyceryl stearate as a mixture with PEG-100 stearate, glyceryl myristate, glyceryl laurate, PEG-40 sorbitan peroleate, laureth-4, ceteareth-3, isostearyl glyceryl ether, cetyl stearyl alcohol as a mixture with sodium cetyl stearyl sulfate, laureth-23, steareth-2, glyceryl stearate as a mixture with PEG-30 stearate, PEG-40 stearate, glycol distearate, PEG-22 dodecyl glycol copolymer, polyglyceryl-2 PEG-4 stearate, ceteareth-12, ceteareth-20, ceteareth-30, methyl glucose sesquistearate, steareth-10, PEG-20 stearate, steareth-2 as a mixture with PEG-8 distearate, steareth-21, steareth-20, isosteareth-20, PEG-45/dodecyl glycol copolymer, methoxy-PEG-22/dodecyl glycol copolymer, PEG-40 sorbitan peroleate, PEG-40 sorbitan perisostearate, PEG-20 glyceryl stearate, PEG-20 glyceryl stearate, PEG-8 beeswax, polyglyceryl 2-laurate, isostearyl diglyceryl succinate, stearamidopropyl-PG dimonium chloride phosphate, glyceryl stearate SE, ceteth-20, triethyl citrate, PEG-20 methyl glucose sesquistearate, glyceryl stearate citrate, cetyl phosphate, cetearyl sulfate, sorbitan sesquioleate, triceteareth 4-phosphate, trilaureth 4-phosphate, polyglyceryl methylglucose distearate, potassium cetyl phosphate, isostearate-10, polyglyceryl 2-sesquiisostearate, ceteth-10, oleth-20, isoceteth-20, glyceryl stearate as a mixture with ceteareth-20, ceteareth-12, cetyl stearyl alcohol and cetyl palmitate, cetyl stearyl alcohol as a mixture with PEG-20 stearate, PEG-30 stearate, PEG-40 stearate, PEG-100 stearate.

It is advantageous according to the invention to choose the weight ratios of coemulsifier A to emulsifier B to emulsifier C (A:B:C) as a:b:c, where a, b and c independently of one another can be rational numbers from 1 to 5, preferably from 1 to 3. A weight ratio of approximately 1:2:1 is particularly preferred.

It is advantageous within the meaning of the present invention to choose the total amount of the emulsifiers A, B and C from the range from 0.1 to 15% by weight, advantageously from 0.5 to 10% by weight, in particular from 2 to 10% by weight, in each case based on the total weight of the formulation.

Silicone Oils

It is preferred to choose the oil phase of the preparations according to the invention to at least 2.0% by weight, based on the total weight of the preparations, from the group of the cyclic and/or linear silicones, which in the context of the present disclosure are also designated as “silicone oils”. Such silicones or silicone oils can be present as monomers, which as a rule are characterized by structural elements as follows:

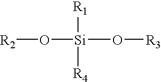

Linear silicones to be employed advantageously according to the invention having a number of siloxyl units are in general characterized by the following structural element: 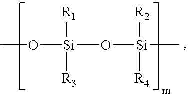
where the silicon atoms can be substituted by identical or different alkyl radicals and/or aryl radicals, which are represented here in general terms by the radicals R₁-R₄ (i.e., the number of the different radicals is not necessarily restricted to up to 4). m can in this case assume values from 2-200,000.

Cyclic silicones to be employed advantageously according to the invention are in general characterized by the following structural element 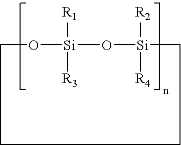
where the silicon atoms can be substituted by identical or different alkyl radicals and/or aryl radicals, which are represented here in general terms by the radicals R₁-R₄ (i.e., the number of the different radicals is not necessarily restricted to up to 4). n can in this case assume values from 3/2 to 20. Fractional values for n take into consideration that odd-numbered numbers of siloxyl groups can be present in the cycle.

Advantageously, phenyl trimethicone is chosen as the silicone oil. Other silicone oils, such as, for example, dimethicone, phenyl dimethicone, cyclomethicone (for example hexamethylcyclotrisiloxane, octamethylcyclotetrasiloxane, cyclopentasiloxane, cyclo-hexasiloxane, and mixtures of these components), polydimethylsiloxane, poly-(methylphenylsiloxane), cetyl dimethicone, behenoxy dimethicone can also be used advantageously within the meaning of the present invention. Mixtures of cyclo-methicone and isotridecyl isononanoate, and those of cyclomethicone and 2-ethyl-hexyl isostearate are furthermore advantageous.

It is, however, also advantageous to choose silicone oils of similar constitution to the above-designated compounds, whose organic side chains are derivatized, for example polyethoxylated and/or polypropoxylated. These include, for example, polysiloxane-polyalkyl-polyether copolymers such as cetyl dimethicone copolyol, (cetyl dimethicone copolyol (and) polyglyceryl 4-isostearate (and) hexyl laurate).

Advantageously, cyclomethicone is employed as the silicone oil to be used according to the invention. However, other silicone oils can also be used advantageously within the meaning of the present invention, for example dimethicone (polydimethyl-siloxane) and also phenyl trimethicone or combinations of the substances mentioned here.

It is advantageous within the meaning of the present invention to restrict the total amount of the silicone oils to 2 to 25% by weight. According to the invention, a total amount of the silicone oils of 5 to 20% by weight and very particularly a total amount of 10 to 15% by weight—always based on the total amount—is particularly advantageous.

Advantageously, the oil phase can further contain cyclic or linear silicone oils or consist completely of such oils, where, however, it is preferred to use, aside from the silicone oil or the silicone oils, an additional content of other oil-phase components.

Oil Phase/Lipids

The oil phase of the formulations according to the invention is advantageously chosen from polar oils, for example from lecithins and fatty acid triglycerides, especially the triglycerol esters of saturated and/or unsaturated, branched and/or unbranched alkanecarboxylic acids of a chain length of 8 to 24, in particular 12 to 18, carbon atoms. The fatty acid triglycerides can, for example, be chosen advantageously from synthetic, semisynthetic and natural oils, such as, for example, coconut glyceride, olive oil, sunflower oil, soybean oil, peanut oil, rapeseed oil, almond oil, palm oil, coconut oil, castor oil, wheatgerm oil, grapeseed oil, thistle oil, evening primrose oil, macadamia nut oil and the like.

Further advantageous polar oil components can be chosen within the meaning of the present invention further from the esters of saturated and/or unsaturated, branched and/or unbranched alkanecarboxylic acids of a chain length of 3 to 30 carbon atoms and saturated and/or unsaturated, branched and/or unbranched alcohols of a chain length of 3 to 30 carbon atoms, and from the esters of aromatic carboxylic acids and saturated and/or unsaturated, branched and/or unbranched alcohols of a chain length of 3 to 30 carbon atoms. Such ester oils can then advantageously be chosen from octyl palmitate, octyl cocoate, octyl isostearate, octyl dodecyl myristate, cetearyl isononanoate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, stearoyl heptanoate, oleyl oleate, oleyl erucate, erucyl oleate, erucyl erucate, tridecyl stearate, tridecyl trimellitate, and also synthetic, semisynthetic and natural mixtures of such esters, such as, for example, jojoba oil.

Furthermore, the oil phase can be advantageously chosen from dialkyl ethers and dialkyl carbonates; for example, dicaprylyl ether (Cetiol OE) and/or dicaprylyl carbonate, for example that obtainable under the trade name Cetiol CC from Cognis, are advantageous.

It is further preferred the oil component(s) from isoeicosane, neopentyl glycol diheptanoate, propylene glycol dicaprylate/dicaprate, caprylic/capric/diglyceryl succinate, butylene glycol dicaprylate/dicaprate, C₁₂₋₁₃-alkyl lactate, di-C₁₂₋₁₃-alkyl tartrate, triisostearin, dipentaerithrityl hexacaprylate/hexa-caprate, propylene glycol monoisostearate, tricaprylin, dimethyl isosorbide. It is particularly advantageous if the oil phase of the formulations according to the invention contains C₁₂₋₁₅-alkyl benzoate or consists completely of this.

Any desired mixtures of such oil and wax components can also be employed advantageously within the meaning of the present invention.

Furthermore, the oil phase can likewise advantageously also contain nonpolar oils, for example those which are chosen from branched and unbranched hydrocarbons and hydrocarbon waxes, in particular mineral oil, petroleum jelly (petrolatum), paraffin oil, squalane and squalene, polyolefins, hydrogenated polyisobutenes and isohexadecane. Among the polyolefins, polydecenes are the preferred substances.

The lipid(s) are chosen according to the invention from the natural and/or synthetic lipids. The following are preferably used: C₁₂-C₁₅ alkyl benzoate, capric/caprylic triglyceride, butylene glycol dicaprylate/dicaprate, octyl dodecanol, dicaprylyl carbonate, dicaprylyl carbonate, dicaprylyl ether, mineral oil, coconut glycerides.

Mixtures of cyclomethicone, dicaprylyl carbonate and C₁₂₋₁₅-alkyl benzoate and of cyclomethicone, dimethicone, butylene glycol dicaprylate/dicaprate, dicaprylyl carbonate and mineral oil are furthermore particularly advantageous.

Advantageously, the content of the fatty phase is between 1 and 80% by weight, based on the total weight of the preparations, preferably 2.5-70% by weight, in particular 5-60% by weight.

Water Phase

The aqueous phase of the preparations according to the invention optionally advantageously contains alcohols, diols or polyols of low carbon number, and also their ethers, preferably ethanol, isopropanol, propylene glycol, glycerol, butylene glycol, ethylene glycol, ethylhexyl glycerol, ethylene glycol monoethyl or monobutyl ether, propylene glycol monomethyl, monoethyl or monobutyl ether, diethylene glycol monomethyl or monoethyl ether and analogous products, furthermore alcohols of low carbon number, e.g. ethanol, isopropanol, 1,2-propanediol, glycerol and also in particular one or more thickening agents which can advantageously be chosen from the group consisting of silicon dioxide, aluminum silicates, polysaccharides and their derivatives, e.g. hyaluronic acid, xanthan gum, hydroxypropylmethylcellulose, particularly advantageously from the group consisting of the polyacrylates, preferably a polyacrylate from the group consisting of the “carbopols”, for example carbopols of the types 980, 981, 1382, 2984, 5984, in each case individually or in combination.

Advantageous preservatives within the meaning of the present invention are, for example, formaldehyde-cleaving agents (such as, for example, DMDM hydantoin [e.g. Glydant®]), iodopropyl butylcarbamate (e.g. obtainable under the trade names Glycacil-L or Glycacil-L and Konkaben LMB from Lonza), parabens, phenoxy-ethanol, ethanol, benzoic acid and suchlike. Customarily, according to the invention the preservative system advantageously also contains preservation aids, such as, for example, ethylhexyloxyglycerol, Glycine soja etc.

In addition, humectants or “moisturizers” can be present.

Moisturizers are designated as substances or substance mixtures which impart to cosmetic or dermatological preparations the property, after the application to or dispersion on the skin surface, of reducing the release of moisture from the horny layer (also called trans-epidermal water loss (TEWL)) and/or of positively influencing the hydration of the horny layer.

Advantageous moisturizers within the meaning of the present invention are, for example, glycerol, lactic acid, pyrrolidonecarboxylic acid and urea. Furthermore, it is particularly advantageous to use polymeric moisturizers from the group consisting of the polysaccharides which are water-soluble and/or swellable in water and/or gellable with the aid of water. Those particularly advantageous are, for example, hyaluronic acid, chitosan and/or a fucose-rich polysaccharide, which is deposited in the Chemical Abstracts under the registry number 178463-23-5 and is obtainable, for example, under the name Fucogel® 1000 from SOLABIA S.A.

The cosmetic or dermatological preparations according to the invention can furthermore advantageously, even though not compulsorily, contain fillers which, for example, further improve the sensory and cosmetic properties of the formulations and, for example, produce or increase a velvety or silky skin sensation. Advantageous fillers within the meaning of the present invention are starch and starch derivatives (such as, for example, tapioca starch, distarch phosphate, aluminum or sodium starch octenylsuccinate and the like), pigments which have neither mainly UV filter nor coloring action (such as, for example, boron nitride etc.) and/or Aerosils® (CAS No. 7631-86-9).

Excipients

The compositions according to the invention can further optionally contain additives customary in cosmetics, for example perfume, thickeners, deodorants, antimicrobial substances, refatting agents, complexing and sequestering agents (e.g. EDTA, imino disuccinic acid), pearl luster agents, plant extracts, vitamins, active ingredients, preservatives, bactericides, colorants, pigments which have a coloring action, thickening agents, moisturizing and/or humectant substances, fats, oils, waxes or other customary constituents of a cosmetic or dermatological formulation such as alcohols, polyols, polymers, foam stabilizers, electrolytes, organic solvents or silicone derivatives.

Dyes

The cosmetic and dermatological preparations according to the invention can contain dyes and/or color pigments, in particular if they are present in the form of decorative cosmetics. The dyes and color pigments can be selected from the corresponding positive list of the Cosmetics Order or the EC list of cosmetic dyes. In most cases, they are identical to the dyes permitted for foodstuffs.

Advantageous color pigments are, for example, titanium dioxide, mica, iron oxides (e.g. Fe₂O₃, Fe₃O₄, FeO(OH)) and/or tin oxide.

Advantageous dyes are, for example, carmine, Prussian blue, chromic oxide green, ultramarine blue and/or manganese violet. It is particularly advantageous to choose the dyes and/or color pigments from the following list. The Colour Index numbers (CIN) are taken from the Rowe Colour Index, 3rd edition, Society of Dyers and Colourists, Bradford, England, 1971.

| Chemical or other name | CIN | Colour |
|---|---|---|
| Pigment Green | 10006 | green |
| Acid Green 1 | 10020 | green |
| 2,4-Dinitrohydroxynaphthalene-7-sulfo acid | 10316 | yellow |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2-hydroxynaphthalene | 12085 | red |
| Pigment Red 3 | 12120 | red |
| Ceresrot; Sudan Red; Fettrot G | 12150 | red |
| Pigment Red 112 | 12370 | red |
| Pigment Red 7 | 12420 | red |
| Pigment Brown 1 | 12480 | brown |
| 4-(2′-Methoxy-5′-sulfo acid diethylamide-1′-phenylazo)-3- | 12490 | red |
| hydroxy-5″-chloro-2″,4″-dimethoxy-2-naphthoic acid anilide | | |
| Disperse Yellow 16 | 12700 | yellow |
| 1-(4-Sulfo-1-phenylazo)-4-aminobenzene-5-sulfo acid | 13015 | yellow |
| 2,4-Dihydroxyazobenzene-4′-sulfo acid | 14270 | orange |
| 2-(2,4-Dimethylphenylazo-5-sulfo acid)-1-hydroxynaphthalene- | 14700 | red |
| 4-sulfo acid | | |
| 2-(4-Sulfo-1-naphthylazo)-1-naphthol-4-sulfo acid | 14720 | red |
| 2-(6-Sulfo-2,4-xylylazo)-1-naphthol-5-sulfo acid | 14815 | red |
| 1-(4′-Sulfophenylazo)-2-hydroxynaphthalene | 15510 | orange |
| 1-(2-Sulfo acid-4-chloro-5-carboxylic acid-1-phenylazo)- | 15525 | red |
| 2-hydroxynaphthalene | | |
| 1-(3-Methylphenylazo-4-sulfo acid)-2-hydroxynaphthalene | 15580 | red |
| 1-(4′,(8′)-Sulfo acid naphthylazo)-2-hydroxynaphthalene | 15620 | red |
| 2-Hydroxy-1,2′-azonaphthalene-1′-sulfo acid | 15630 | red |
| 3-Hydroxy-4-phenylazo-2-naphthylcarboxylic acid | 15800 | red |
| 1-(2-Sulfo-4-methyl-1-phenylazo)-2-naphthylcarboxylic acid | 15850 | red |
| 1-(2-Sulfo-4-methyl-5-chloro-1-phenylazo)-2-hydroxy- | 15865 | red |
| naphthalene-3-carboxylic acid | | |
| 1-(2-Sulfo-1-naphthylazo)-2-hydroxynaphthalene-3-carboxylic | 15880 | red |
| acid | | |
| 1-(3-Sulfo-1-phenylazo)-2-naphthol-6-sulfo acid | 15980 | orange |
| 1-(4-Sulfo-1-phenylazo)-2-naphthol-6-sulfo acid | 15985 | yellow |
| Allura Red | 16035 | red |
| 1-(4-Sulfo-1-naphthylazo)-2-naphthol-3,6-disulfo acid | 16185 | red |
| Acid Orange 10 | 16230 | orange |
| 1-(4-Sulfo-1-naphthylazo)-2-naphthol-6,8-disulfo acid | 16255 | red |
| 1-(4-Sulfo-1-naphthylazo)-2-naphthol-3,6,8-trisulfo acid | 16290 | red |
| 8-Amino-2-phenylazo-1-naphthol-3,6-disulfo acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5-hydroxypyrazolone- | 19140 | yellow |
| 3-carboxylic acid | | |
| Pigment Yellow 16 | 20040 | yellow |
| 2,6-(4′-Sulfo-2″,4″-dimethyl)bisphenylazo)1,3-dihydroxy- | 20170 | orange |
| benzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′-naphthylazo]-1-hydroxy- | 27755 | black |
| 7-aminonaphthalene-3,6-disulfo acid | | |
| 4′-[(4″-Sulfo-1″-phenylazo)-7′-sulfo-1′-naphthylazo]-1-hydroxy-8- | 28440 | black |
| acetylaminonaphthalene-3,5-disulfo acid | | |
| Direct Orange 34, 39, 44, 46, 60 | 40215 | orange |
| Food Yellow | 40800 | orange |
| trans-β-Apo-8′-carotenal (C₃₀) | 40820 | orange |
| trans-Apo-8′-carotenic acid (C₃₀)-ethyl ester | 40825 | orange |
| Canthaxanthin | 40850 | orange |
| Acid Blue 1 | 42045 | blue |
| 2,4-Disulfo-5-hydroxy-4′-4″-bis(diethylamino)triphenylcarbinol | 42051 | blue |
| 4-[(-4-N-Ethyl-p-sulfobenzylamino)phenyl-(4-hydroxy-2-sulfo- | 42053 | green |
| phenyl)(methylene)-1-(N-ethyl-N-p-sulfobenzyl)-2,5-cyclohexa- | | |
| dienimine] | | |
| Acid Blue 7 | 42080 | blue |
| (N-Ethyl-p-sulfobenzylamino)phenyl-(2-sulfophenyl)methylene- | 42090 | blue |
| (N-ethyl-N-p-sulfo-benzyl)-Δ^{2,5}-cyclohexadienimine | | |
| Acid Green 9 | 42100 | green |
| Diethyldisulfobenzyldi-4-amino-2-chlorodi-2-methylfuchson- | 42170 | green |
| immonium | | |
| Basic Violet 14 | 42510 | violet |
| Basic Violet 2 | 42520 | violet |
| 2′-Methyl-4′-(N-ethyl-N-m-sulfobenzyl)amino-4″-(N-diethyl)- | 42735 | blue |
| amino-2-methyl-N-ethyl-(N-m-sulfobenzylfuchsonimmonium | | |
| 4′-(N-Dimethyl)amino-4″-(N-phenyl)aminonaphtho-N-dimethyl- | 44045 | blue |
| fuchsonimmonium | | |
| 2-Hydroxy-3,6-disulfo-4,4′-bis-dimethylaminonaphthofuchson- | 44090 | green |
| immonium | | |
| Acid Red 52 | 45100 | red |
| 3-(2′-Methylphenylamino)-6-(2′-methyl-4′-sulfophenylamino)- | 45190 | violet |
| 9-(2″-carboxyphenyl)xanthenium salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Quinophthalone | 47000 | yellow |
| Quinophthalone disulfo acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium-aluminium complex | 58000 | red |
| 3-Oxypyrene-5,8,10-sulfo acid | 59040 | green |
| 1-Hydroxy-4-N-phenylaminoanthraquinone | 60724 | violet |
| 1-Hydroxy-4-(4′-methylphenylamino)anthraquinone | 60725 | violet |
| Acid Violet 23 | 60730 | violet |
| 1,4-Di(4′-methylphenylamino)anthraquinone | 61565 | green |
| 1,4-Bis-(o-Sulfo-p-toluidino)anthraquinone | 61570 | green |
| Acid Blue 80 | 61585 | blue |
| Acid Blue 62 | 62045 | blue |
| N,N′-Dihydro-1,2,1′,2′-anthraquinazine | 69800 | blue |
| Vat Blue 6; Pigment Blue 64 | 69825 | blue |
| Vat Orange 7 | 71105 | orange |
| Indigo | 73000 | blue |
| Indigo disulfo acid | 73015 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanine | 74260 | green |
| Natural Yellow 6, 19; Natural Red 1 | 75100 | yellow |
| Bixin, norbixin | 75120 | orange |
| Lycopene | 75125 | yellow |
| trans-alpha-, beta- or gamma-Carotene | 75130 | orange |
| Keto- and/or hydroxyl derivatives of Carotene | 75135 | yellow |
| Guanine or pearl luster agent | 75170 | white |
| 1,7-Bis-(4-hydroxy-3-methoxyphenyl)-1,6-heptadiene-3,5-dione | 75300 | yellow |
| Complex salt (Na, Al, Ca) of carminic acid | 75470 | red |
| Chlorophyll a and b; copper compounds of chlorophylls and | 75810 | green |
| chlorophyllins | | |
| Aluminum | 77000 | white |
| Aluminum hydroxide | 77002 | white |
| Water-containing aluminum silicates | 77004 | white |
| Ultramarine | 77007 | blue |
| Pigment Red 101 and 102 | 77015 | red |
| Barium sulfate | 77120 | white |
| Bismuth oxychloride and its mixtures with mica | 77163 | white |
| Calcium carbonate | 77220 | white |
| Calcium sulfate | 77231 | white |
| Carbon | 77266 | black |
| Pigment Black 9 | 77267 | black |
| Carbo medicinalis vegetabilis | 77268:1 | black |
| Chromic oxide | 77288 | green |
| Chromic oxide; water-containing | 77289 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxide | 77491 | red |
| Ferric hydroxide | 77492 | yellow |
| Iron oxide | 77499 | black |
| Mixtures of iron(II) and iron(III) hexacyanoferrate | 77510 | blue |
| Pigment White 18 | 77713 | white |
| Manganese ammonium diphosphate | 77742 | violet |
| Manganese phosphate; Mn₃(PO₄)₂ · 7 H₂O | 77745 | red |
| Silver | 77820 | white |
| Titanium dioxide and its mixtures with mica | 77891 | white |
| Zinc oxide | 77947 | white |
| 6,7-Dimethyl-9-(1′-D-ribityl)-isoalloxazine, lactoflavin | | yellow |
| Caramel | | brown |
| Capsanthin, capsorubicin | | orange |
| Betanin | | red |
| Benzopyrylium salts, anthocyans | | red |
| Aluminum, zinc, magnesium and calcium stearate | | white |
| Bromothymol blue | | blue |
| Bromocresol green | | green |
| Acid Red 195 | | red |

If the formulations according to the invention are present in the form of products which are applied to the face, it is convenient to use as a dye one or more substances from the following group: 2,4-dihydroxyazobenzene, 1-(2′-chloro-4′-nitro-1′-phenylazo)-2-hydroxynaphthalene, Ceresrot, 2-(4-sulfo-1-naphthylazo)-1-naphthyl-4-sulfo acid, calcium salt of 2-hydroxy-1,2′-azonaphthalene-1′-sulfo acid, calcium and barium salts of 1-(2-sulfo-4-methyl-1-phenylazo)-2-naphthylcarboxylic acid, calcium salt of 1-(2-sulfo-1-naphthylazo)-2-hydroxynaphthalene-3-carboxylic acid, aluminum salt of 1-(4-sulfo-1-phenylazo)-2-naphthol-6-sulfo acid, aluminum salt of 1-(4-sulfo-1-naphthylazo)-2-naphthol-3,6-disulfo acid, 1-(4-sulfo-1-naphthylazo)-2-naphthol-6,8-disulfo acid, aluminum salt of 4-(4-sulfo-1-phenylazo)-1-(4-sulfo-phenyl)-5-hydroxypyrazolone-3-carboxylic acid, aluminum and zirconium salts of 4,5-dibromofluorescein, aluminum and zirconium salts of 2,4,5,7-tetrabromofluorescein, 3′,4′,5′,6′-tetrachloro-2,4,5,7-tetrabromofluorescein and its aluminum salt, aluminum salt of 2,4,5,7-tetraiodofluorescein, aluminum salt of quinophthalonedisulfo acid, aluminum salt of indigo disulfo acid, red and black iron oxide (CIN: 77 491 (red) and 77 499 (black)), ferric hydroxide (CIN: 77 492), manganese ammonium diphosphate and titanium dioxide.

Oil-soluble natural dyes, such as, for example, paprika extracts, β-carotene or cochineal are furthermore advantageous.

Formulations containing pearl luster pigments are furthermore advantageous within the meaning of the invention. In particular, the types of pearl luster pigments listed below are preferred:
1. Natural pearl luster pigments, such as, for example
   “silver gray” (guanine/hypoxanthine mixed crystals from fish scales) and
   “mother of pearl” (ground mussel shells)
2. Monocrystalline pearl luster pigments such as, for example, bismuth oxychloride (BiOCl)
3. Layer substrate pigments: e.g. mica/metal oxide

Powdered pigments or castor oil dispersions of bismuth oxychloride and/or titanium oxide, and bismuth oxychloride and/or titanium dioxide on mica, for example, are the basis for pearl luster pigments. The luster pigment listed under CIN 77163, for example, is particularly advantageous.

The following pearl luster pigments based on mica/metal oxide, for example, are furthermore advantageous:

| Group | Coating/layer thickness | Color |
|---|---|---|
| Silver-white pearl | TiO₂: 40-60 nm | silver |
| luster pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Color luster pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold shades |
| | TiO₂/Cr₂O₃ | green |
| | TiO₂/Prussian blue | deep blue |
| | TiO₂/carmine | red |

The pearl luster pigments obtainable from Merck under the trade names Timiron, Colorona or Dichrona, for example, are particularly preferred.

The list of the pearl luster pigments mentioned is not intended, of course, to be limiting. Within the meaning of the present invention, advantageous pearl luster pigments are obtainable in numerous ways known per se. For example, other substrates aside from mica can also be coated with further metal oxides, such as, for example, silica and suchlike. SiO₂ particles coated with TiO₂ and Fe₂O₃ (“Ronaspheres”), for example, which are marketed by Merck are advantageous and are particularly suitable for the visual reduction of fine lines.

It can moreover be advantageous to dispense completely with a substrate such as mica. Iron pearl luster pigments which can be prepared without the use of mica are particularly preferred. Such pigments are obtainable from BASF, for example, under the trade names Sicopearl Kupfer 1000.

Effect pigments which are obtainable under the trade name Metasomes standard/ glitter in various colors (yellow, red, green, blue) from Flora Tech are furthermore also particularly advantageous. The glitter particles are present here as mixtures with various excipients and dyes (such as, for example, the dyes having the Colour Index (CI) numbers 19140, 77007, 77289, 77491).

The dyes and pigments can be present either individually or as a mixture, and can also be mutually coated with one another, in general various color effects being produced by means of different coating thicknesses. The total amount of the dyes and color-imparting pigments is advantageously chosen from the range from, for example, 0.1% by weight to 30% by weight, preferably from 0.5 to 15% by weight, in particular from 1.0 to 10% by weight, in each case based on the total weight of the preparations.

Active Ingredients

Particularly advantageous preparations are further obtained if antioxidants are employed as additives or active ingredients. According to the invention, the preparations advantageously contain one or more antioxidants. As convenient antioxidants, which, however, are nevertheless to be used optionally, it is possible to use all antioxidants which are suitable or customary for cosmetic and/or dermatological applications.

Advantageously, the antioxidants are chosen from amino acids (e.g. glycine, histidine, tyrosine, tryptophan) and their derivatives, imidazoles (e.g. urocaninic acid) and its derivatives, peptides such as D,L-carnosine, D-carnosine, L-carnosine and their derivatives (e.g. anserine), carotenoids, carotenes (e.g. α-carotene, β-carotene, lycopene) and their derivatives, lipoic acid and its derivatives (e.g. dihydrolipoic acid), aurothioglucose, propylthiouracil and other thiols (e.g. thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters), and their salts, dilauryl thiodipropionate, distearyl thiodipropionate, thiodipropionic acid and its derivatives (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerable doses (e.g. pmol to μmol/kg), furthermore (metal) chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. γ-linolenic acid, linoleic acid, oleic acid), folic acid and its derivatives, ubiquinone and ubiquinol and their derivatives, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutic acid and its derivatives, ferulic acid and its derivatives, butylhydroxytoluene, butylhydroxy-anisole, nordihydroguaiaretic acid, nordihydroguaiaretic acid, trihydroxy-butyrophenone, uric acid and its derivatives, mannose and its derivatives, zinc and its derivatives (e.g. ZnO, ZnSO₄), selenium and its derivatives (e.g. selenomethionine), stilbenes and their derivatives (e.g. stilbene oxide, trans-stilbene oxide) and the derivatives suitable according to the invention (salts, esters, ethers, sugars, nucleotides, nucleosides, peptides and lipids) of these mentioned active ingredients.

Water-soluble antioxidants can be employed particularly advantageously within the meaning of the present invention, such as, for example, vitamins, e.g. ascorbic acid or tocopherol and their derivatives.

A surprising property of the preparations according to the invention is that they are very good vehicles for cosmetic or dermatological active ingredients in the skin, preferred active ingredients being antioxidants which can protect the skin from oxidative stress. Preferred antioxidants are in this case vitamin E and its derivatives, and vitamin A and its derivatives.

The amount of the antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or its derivatives is/are the antioxidant(s), it is advantageous to choose their respective concentrations from the range from 0.001 to 10% by weight, based on the total weight of the formulation.

If vitamin A or vitamin A derivatives, or carotenes or their derivatives is/are the antioxidant (s), it is advantageous to choose their respective concentrations from the range from 0.001 to 10% by weight, based on the total weight of the formulation.

According to the invention, the active ingredients (one or more compounds) can also very advantageously be chosen from lipophilic active ingredients, in particular from the following group: acetylsalicylic acid, atropine, azulene, hydrocortisone and its derivatives, e.g. hydrocortisone 17-valerate, vitamins of the B and D series, very favorably vitamin B₁, vitamin B₁₂, vitamin D₁, but also bisabolol, unsaturated fatty acids, especially the essential fatty acids (often also called vitamin F), in particular gamma-linolenic acid, oleic acid, eicosapentaenoic acid, docosahexaenoic acid and their derivatives, chloramphenicol, caffeine, prostaglandins, thymine, camphor, extracts or other products of vegetable and animal origin, e.g. evening primrose oil, borage oil or currant pip oil, fish oils, cod-liver oil but also ceramides and ceramide-like compounds etc.

It is also advantageous to choose the active ingredients from the group consisting of the refatting substances, for example purcellin oil, Eucerit® and Neocerit®.

Particularly advantageously, the active ingredient(s) are further chosen from the group consisting of the NO synthase inhibitors, in particular if the preparations according to the invention are to be used for the treatment and prophylaxis of the symptoms of intrinsic and/or extrinsic skin ageing, and for the treatment and prophylaxis of the harmful effects of ultraviolet radiation on the skin.

A preferred NO synthase inhibitor is nitroarginine.

Additionally advantageously, the active ingredient(s) are chosen from the group which includes catechols and bile acid esters of catechols and aqueous or organic extracts of plants or plant parts which contain the catechols or bile acid esters of catechols, such as, for example, the leaves of the plant family Theaceae, in particular of the species Camellia sinensis (green tea). Their typical ingredients (such as, for example, polyphenols or catechols, caffeine, vitamins, sugars, minerals, amino acids, lipids) are particularly advantageous.

Catechols are a group of compounds which are to be interpreted as hydrogenated flavones or anthocyanidines and derivatives of “catechol” (3,3′,4′,5,7-flavanpentaol, 2-(3,4-dihydroxyphenyl)chroman-3,5,7-triol). Epicatechol ((2R,3R)-3,3′,4′,5,7-flavan-pentaol) is also an advantageous active ingredient within the meaning of the present invention.

Plant extracts containing catechols, in particular extracts of green tea, such as, for example, extracts of leaves of the plants of the species Camellia spec., very particularly of the tea species Camellia sinensis, C. assamica, C. taliensis or C. irrawadiensis and crossings of these with, for example, Camellia japonica are furthermore advantageous.

Preferred active ingredients are furthermore polyphenols or catechols from the group consisting of (−)-catechol, (+)-catechol, (−)-catechol gallate, (−)-gallocatechol gallate, (+)-epicatechol, (−)-epicatechol, (−)-epicatechol gallate, (−)-epigallocatechol, (−)-epigallocatechol gallate.

Flavone and its derivatives (often also collectively called “flavones”) are also advantageous active ingredients within the meaning of the present invention. They are characterized by the following basic structure (substitution positions indicated):

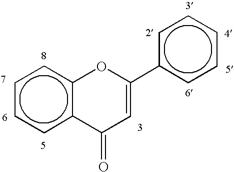

Some of the more important flavones, which can also preferably be employed in the preparations according to the invention, are listed in the table below:

| | OH substitution positions | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Campherol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones as a rule occur in glycosidated form.

According to the invention, the flavonoids are preferably chosen from substances of the generic structural formula 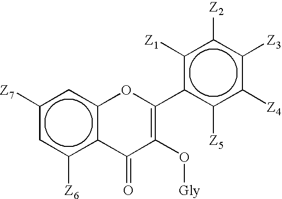
where Z₁ to Z₇ independently of one another are chosen from the group consisting of H, OH, alkoxy and hydroxyalkoxy groups, where the alkoxy or hydroxyalkoxy groups can be branched and unbranched and can have 1 to 18 carbon atoms, and where Gly is chosen from the group consisting of the mono- and oligoglycoside radicals.

According to the invention, the flavonoids, however, can also advantageously be chosen from substances of the generic structural formula 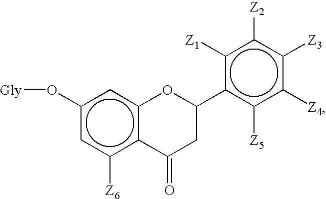
where Z₁ to Z₆ independently of one another are chosen from the group consisting of H, OH, alkoxy and hydroxyalkoxy groups, where the alkoxy or hydroxyalkoxy groups can be branched and unbranched and can have 1 to 18 carbon atoms, and where Gly is chosen from the group consisting of the mono- and oligoglycoside radicals.

Preferably, such structures can be chosen from substances of the generic structural formula 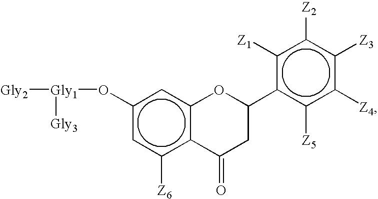
where Gly₁, Gly₂ and Gly₃ independently of one another are monoglycoside radicals or. Gly₂ and Gly₃ can also individually or together be saturations by hydrogen atoms.

Preferably, Gly₁, Gly₂ and Gly₃ independently of one another are chosen from hexosyl radicals, in particular the rhamnosyl radicals and glucosyl radicals. However, other hexosyl radicals, for example allosyl, altrosyl, galactosyl, gulosyl, idosyl, mannosyl and talosyl can optionally also be used advantageously. It can also be advantageous according to the invention to use pentosyl radicals.

Advantageously, Z₁ to Z₅ independently of one another are chosen from the group consisting of H, OH, methoxy, ethoxy and 2-hydroxyethoxy groups, and the flavone glycosides have the structure

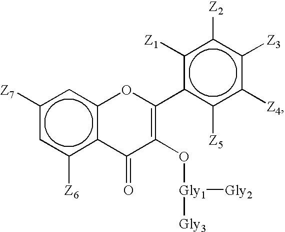

Particularly advantageously, the flavone glycosides according to the invention are from the group which are represented by the following structure: 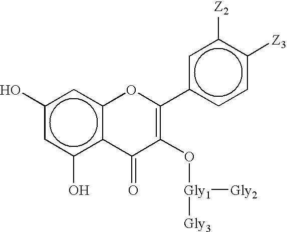
where Gly₁, Gly₂ and Gly₃ independently of one another are monoglycoside radicals or. Gly₂ and Gly₃ can also individually or together be saturations by hydrogen atoms.

Preferably, Gly₁, Gly₂ and Gly₃ independently of one another are chosen from hexosyl radicals, in particular the rhamnosyl radicals and glucosyl radicals. However, other hexosyl radicals, for example allosyl, altrosyl, galactosyl, gulosyl, idosyl, mannosyl and talosyl can optionally also be used advantageously. It can also be advantageous according to the invention to use pentosyl radicals.

It is particularly advantageous within the meaning of the present invention to choose the flavone glycoside(s) from α-glucosylrutin, α-glucosyl-myricetin, α-glucosylisoquercitrin, α-glucosylisoquercetin and α-glucosylquercitrin.

α-Glucosylrutin is particularly preferred according to the invention.

Naringin (aurantiin, naringenin 7-rhamnoglucoside), hesperidin (3′,5,7-trihydroxy-4′-methoxyflavanone 7-rutinoside, hesperidoside, hespereitin 7-O-rutinoside), rutin (3,3′,4′,5,7-pentahydroxyflyvone 3-rutinoside, quercetin 3-rutinoside, sophorin, Birutan, Rutabion, taurutin, phytomelin, melin), troxerutin (3,5-dihydroxy-3′,4′,7-tris(2-hydroxyethoxy)flavone 3-(6-O-(6-deoxy-α-L-mannopyranosyl)-β-D-glucopyranoside)), monoxerutin (3,3′,4′,5-tetrahydroxy-7-(2-hydroxyethoxy)flavone-3-(6-O-(6-deoxy-α-L-mannopyranosyl)-β-D-glucopyranoside)), dihydrorobinetin (3,3′,4′,5′,7-pentahydroxy-flavanone), taxifolin (3,3′,4′,5,7-pentahydroxyflavanone), eriodictyol 7-glucoside (3′,4′,5,7-tetrahydroxyflavanone 7-glucoside), flavanomarein (3′,4′,7,8-tetrahydroxy-flavanone 7-glucoside) and isoquercetin (3,3′,4′,5,7-pentahydroxyflavanone 3-(β-D-glucopyranoside) are also advantageous according to the invention.

It is also advantageous to choose the active ingredient(s) from the group consisting of the ubiquinones and plastoquinones.

Ubiquinones are distinguished by the structural formula 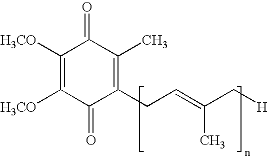
and are the most widespread and thus the best investigated bioquinones. Depending on the number of the isoprene units linked in the side chain, ubiquinones are called Q-1, Q-2, Q-3, etc or according to the number of carbon atoms U-5, U-10, U-15 etc. They preferably occur with certain chain lengths, e.g. in some microorganisms and yeasts with n=6. Q 10 predominates in most mammals including man.

Coenzyme Q10, which is characterized by the following structural formula 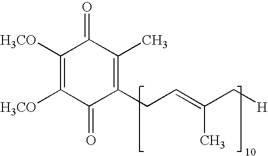
is particularly advantageous.

Plastoquinones have the general structural formula

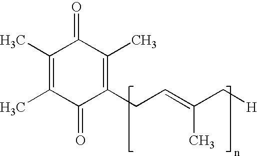

Plastoquinones are distinguished in the number n of the isoprene radicals and are named accordingly, e.g. PQ-9 (n=9). Other plastoquinones with different substituents on the quinone ring additionally exist.

Creatine and/or creatine derivatives are also preferred active ingredients within the meaning of the present invention. Creatine is distinguished by the following structural formula:

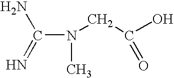

Preferred derivatives are creatine phosphate, and creatine sulfate, creatine acetate, creatine ascorbate and the derivatives esterified on the carboxyl group by mono- or polyfunctional alcohols.

A further advantageous active ingredient is L-carnitine [3-hydroxy-4-(trimethyl-ammonio)butyric acid betaine]. Acylcarnitines, which are chosen from substances of the following general structural formula 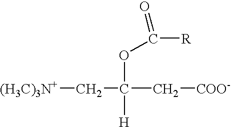
where R is chosen from the group consisting of the branched and unbranched alkyl radicals having up to 10 carbon atoms are advantageous active ingredients within the meaning of the present invention. Propionylcarnitine and in particular acetylcarnitine are preferred. Both enantiomers (D- and L-form) can be used advantageously within the meaning of the present invention. It can also be advantageous to use any desired mixture of enantiomers, for example a racemate of the D- and L-form.

Further advantageous active ingredients are sericoside, pyridoxol, vitamin K, biotin and aromatic substances.

The list of active ingredients or active ingredient combinations mentioned which can be used in the preparations according to the invention is not intended, of course, to be limiting. The active ingredients can be used individually or in any desired combinations with one another.

Moreover, selected formulations according to the invention which, for example, contain known antiwrinkle active ingredients such as flavone glycosides (in particular α-glycosylrutin), coenzyme Q10, vitamin E and/or derivatives and the like, are particularly advantageously suitable for the prophylaxis and treatment of cosmetic or dermatological skin changes, such as occur, for example, on ageing of the skin. They are furthermore advantageous against the syndrome of dry or rough skin.

Skin ageing is caused, for example, by endogenous, genetically determined factors. In the epidermidis and dermis, age-related disturbances, e.g. the following structural damage and functional disturbances occur, which can also come under the term “senile xerosis”:
a) dryness, roughness and formation of (dryness) lines,
b) itching and
c) decreased refatting by sebaceous glands (e.g. after washing).

Exogenous factors, such as UV light and chemical noxae, can have a cumulative action and, for example, accelerate the endogenous ageing processes or supplement them. In the epidermidis and dermis, the following structural damage and functional disturbances, for example, in particular occur in the skin due to exogenous factors, which extend beyond the extent and quality of the damage in the case of chronological ageing:
d) visible vasodilatation (teleangiectasies, cuperosis);
e) flabbiness and formation of lines;
f) local hyper-, hypo- and malpigmentation (e.g. age spots) and
g) increased susceptibility to mechanical stress (e.g. fissurability).

In a particular embodiment, the present invention relates in particular to products for the care of naturally aged skin, and for the treatment of the subsequent damage due to light ageing, in particular the phenomena mentioned under a) to g).

### Specific Application

The cosmetic and/or dermatological preparations according to the invention can have the customary composition and be used for cosmetic and/or dermatological light protection, further for the treatment, the care and the cleansing of the skin and/or the hair and as make-up products in decorative cosmetics.

For application, the cosmetic and dermatological preparations according to the invention are applied to the skin and/or the hair in adequate amounts in the manner customary for cosmetics.

Protection Against the Sun

A further advantageous embodiment of the present invention consists in products for protection against the sun.

An addition of oil-soluble and/or water-soluble and/or pigmentary organic UV filters and/or inorganic pigments absorbing or reflecting UV radiation is particularly advantageous.

It is also advantageous within the meaning of the present invention to make available cosmetic and dermatological preparations whose main aim is not protection from sunlight, but which, nevertheless, can contain UV protection substances. Thus UV-A or UV-B filter substances are usually incorporated, for example, into day creams or make-up products. The UV protection substances, just like antioxidants and, if desired, preservatives, also represent an effective protection of the preparations themselves against deterioration. Cosmetic and dermatological preparations which are present in the form of a sunscreen are furthermore favorable.

The formulations can optionally, although not necessarily, also contain one or more organic and/or inorganic pigments as UV filter substances, which can be present in the water and/or the oil phase.

Preferred inorganic pigments are metal oxides and/or other metal compounds which are poorly soluble or insoluble in water, in particular oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides.

Within the meaning of the present invention, such pigments can advantageously be surface-treated (“coated”), where, for example, an amphiphilic or hydrophobic character is to be formed or retained. This surface treatment can consist in providing the pigments with a thin hydrophobic layer by processes known per se.

The titanium dioxide pigments can be present both in the crystal modification rutile and anatase and can advantageously be surface-treated (“coated”) within the meaning of the present invention, where, for example, a hydrophilic, amphiphilic or hydrophobic character is to be formed or retained. This surface treatment can consist in treating the pigments with a thin hydrophilic and/or hydrophobic inorganic and/or or organic layer by processes known per se. The various surface coating can within the meaning of the present invention also contain water.

Inorganic surface coatings within the meaning of the present invention can consist of aluminum oxide (Al₂O₃), aluminum hydroxide Al(OH)₃, or aluminum oxide hydrate (also: alumina CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9) or iron oxide (Fe₂O₃). These inorganic surface coatings can occur on their own, in combination and/or in combination with organic coating materials.

Organic surface coatings within the meaning of the present invention can consist of vegetable or animal aluminum stearate, vegetable or animal stearic acid, lauric acid, dimethylpolysiloxane (also: dimethicone), methylpolysiloxane (methicone), simethicone (a mixture of dimethylpoly-siloxane with an average chain length of 200 to 350 dimethylsiloxane units and silica gel) or alginic acid (algic acid). These organic surface coatings can occur on their own, in combination and/or in combination with inorganic coating materials.

Within the meaning of the present invention, coated and uncoated titanium dioxides described can also be used in the form of commercially obtainable oily or aqueous predispersions. Dispersing aids and/or solubilizers can advantageously be added to these predispersions.

Suitable titanium dioxide particles and predispersions of titanium dioxide particles within the meaning of the present invention are obtainable from the companies mentioned under the following trade names:

| | | Additional | |
|---|---|---|---|
| | Coating/ | constituents in | |
| Trade name | surface coating | predispersions | Manufacturer |
| MT-150W | None | — | Tayca |
| | | | Corporation |
| MT-150A | None | — | Tayca |
| | | | Corporation |
| MT-500B | None | — | Tayca |
| | | | Corporation |
| MT-600B | None | — | Tayca |
| | | | Corporation |
| MT-100TV | Aluminum hydroxide | — | Tayca |
| | Stearic acid | | Corporation |
| MT-100Z | Aluminum hydroxide | — | Tayca |
| | Stearic acid | | Corporation |
| MT-100T | Aluminum hydroxide | — | Tayca |
| | Stearic acid | | Corporation |
| MT-500T | Aluminum hydroxide | — | Tayca |
| | Stearic acid | | Corporation |
| MT-100S | Aluminum hydroxide | — | Tayca |
| | Lauric acid | | Corporation |
| MT-100F | Stearic acid | — | Tayca |
| | Iron oxide | | Corporation |
| MT-100SA | Alumina | — | Tayca |
| | Silica | | Corporation |
| MT-500SA | Alumina | — | Tayca |
| | Silica | | Corporation |
| MT-600SA | Alumina | — | Tayca |
| | Silica | | Corporation |
| MT-100SAS | Alumina | — | Tayca |
| | Silica | | Corporation |
| | Silicone | | |
| MT-500SAS | Alumina | — | Tayca |
| | Silica | | Corporation |
| | Silicone | | |
| MT-500 H | Alumina | — | Tayca |
| | | | Corporation |
| MT-100AQ | Silica | — | Tayca |
| | Aluminum hydroxide | | Corporation |
| | Alginic acid | | |
| Eusolex T | Aqua | — | Merck KgaA |
| | Simethicone | | |
| Eusolex | Alumina | — | Merck KgaA |
| T-2000 | Simethicone | | |
| Eusolex | Silica | C₁₂₋₁₅ alkyl- | Merck KgaA |
| T-Olio F | Dimethylsilate | benzoate | |
| | Aqua | Calcium poly- | |
| | | hydroxystearate | |
| | | Silica dimethyl- | |
| | | silate | |
| Eusolex | Aqua | Octyl palmitate | Merck KgaA |
| T-Olio P | Simethicone | PEG-7 | |
| | | hydrogenated | |
| | | castor oil | |
| | | Sorbitan oleate | |
| | | Hydrogenated | |
| | | castor oil | |
| | | Beeswax | |
| | | Stearic acid | |
| Eusolex | Aqua | Phenoxyethanol | Merck KgaA |
| T-Aqua | Alumina | Sodium | |
| | Sodium meta- | methylparabens | |
| | phosphate | Sodium meta- | |
| | | phosphates | |
| Eusolex | Alumina | Isononyl iso- | Merck KgaA |
| T-45D | Simethicone | nonanoate | |
| | | Polyglyceryl | |
| | | ricinoleate | |
| Kronos | None | — | Kronos |
| 1171 | | | |
| (titanium | | | |
| dioxide | | | |
| 171) | | | |
| Titanium | None | — | Degussa |
| dioxide | | | |
| P25 | | | |
| Titanium | Octyltrimethyl- | — | Degussa |
| dioxide | silane | | |
| T 805 | | | |
| (Uvinul | | | |
| TiO₂) | | | |
| UV-Titan | Alumina | — | Kemira |
| X610 | Dimethicone | | |
| UV-Titan | Alumina | — | Kemira |
| X170 | Dimethicone | | |
| UV-Titan | Alumina | — | Kemira |
| X161 | Silica | | |
| | Stearic acid | | |
| UV-Titan | Alumina | — | Kemira |
| M210 | | | |
| UV-Titan | Alumina | Glycerol | Kemira |
| M212 | | | |
| UV-Titan | Alumina | — | Kemira |
| M262 | Silicone | | |
| UV-Titan | Alumina | — | Kemira |
| M160 | Silica | | |
| | Stearic acid | | |
| Tioveil | Alumina | Aqua | Solaveil |
| AQ 10PG | Silica | Propylene glycol | Uniquema |
| Mirasun | Alumina | Aqua | Rhone-Poulenc |
| TiW 60 | Silica | | |

Very particularly advantageous titanium dioxides are Eusolex T-2000 and Eusolex T-aqua from Merck, MT-100 TV and MT-100 Z from Tayca, titanium dioxide T 805 from Degussa and Tioveil AQ 10PG from Solaveil.

A further advantageous coating of the inorganic pigments consists of dimethylpoly-siloxane (also: dimethicone), a mixture of fully methylated, linear siloxane polymers which are terminally blocked with trimethylsiloxy units.

Suitable zinc oxide particles and predispersions of zinc oxide particles within the meaning of the present invention are obtainable from the companies mentioned under the following trade names:

| Trade name | Manufacturer | Coating |
|---|---|---|
| Z-Cote HP1 | BASF | 2% dimethicone |
| Z-Cote | BASF | / |
| ZnO NDM | H & R | 5% dimethicone |
| ZnO neutral | H & R | / |
| MZ-300 | Tayca | / |
| MZ-500 | Tayca | / |
| MZ-700 | Tayca | / |
| MZ-303S | Tayca | 3% methicone |
| MZ-505S | Tayca | 5% methicone |
| MZ-707S | Tayca | 7% methicone |
| MZ-303M | Tayca | 3% dimethicone |
| MZ-505M | Tayca | 5% dimethicone |
| MZ-707M | Tayca | 7% dimethicone |
| Z-Sperse | Collaborative | ZnO (>=56%)/ |
| Ultra | Laboratories | dispersion in dimethicone/ |
| | | cyclomethicone/ethylhexyl |
| | | hydroxystearate benzoate |
| Samt-UFZO- | Miyoshi Kasei | ZnO (60%)/ |
| 450/D5 (60%) | | dispersion in |
| | | cyclomethicone/ |
| | | dimethicone |

Within the meaning of the invention, the zinc oxides Z-Cote and Z-Cote HP1 from BASF, zinc oxide NDM from Haarmann & Reimer, and MZ-505S from Tayca are particularly preferred.

An advantageous organic pigment within the meaning of the present invention is 2,2′-methylenebis(6-(2H-benzotriazol-2-yl)-4-(1,1,3,3-tetramethylbutyl)phenol) [INCI: Bisoctyltriazole], which is characterized by the chemical structural formula 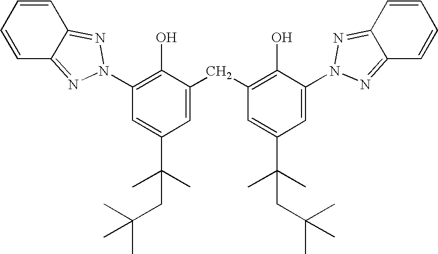
and is obtainable from CIBA Chemikalien GmbH under the trade name Tinosorb® M.

Advantageously, preparations according to the invention contain substances which absorb UV radiation in the UV-A and/or UV-B range, the total amount of the filter substances being, for example, 0.1% by weight to 30% by weight, preferably 0.5 to 20% by weight, in particular 1.0 to 15.0% by weight, based on the total weight of the preparations, in order to make available cosmetic preparations which protect the hair or the skin from the entire range of ultraviolet radiation. They can also be used as a sunscreen for the hair or the skin.

Advantageous further UV-A filter substances within the meaning of the present invention are dibenzoylmethane derivatives, in particular 4-(tert-butyl)-4′-methoxydi-benzoylmethane (CAS No. 70356-09-1), which is marketed by Givaudan under the brand Parsol® 1789 and by Merck under the trade name Eusolex® 9020.

Advantageous sulfonated, water-soluble UV filters within the meaning of the present invention are:
phenylene-1,4-bis(2-benzimidazyl)-3,3′-5,5′-tetrasulfonic acid, which is distinguished by the following structure: 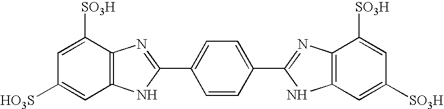
   and its salts, particularly the corresponding sodium, potassium or triethanol-ammonium salts, in particular phenylene-1,4-bis(2-benzimidazyl)-3,3′-5,5′-tetra-sulfonic acid bis sodium salt 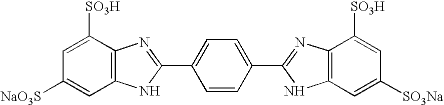
   having the INCI name Bisimidazylate (CAS No.: 180898-37-7), which is obtainable from Haarmann & Reimer, for example, under the trade name Neo Heliopan AP.

A further sulfonated UV filter within the meaning of the present invention are the salts of 2-phenylbenzimidazole-5-sulfonic acid, such as their sodium, potassium or their triethanol ammonium salts, and the sulfonic acid itself. 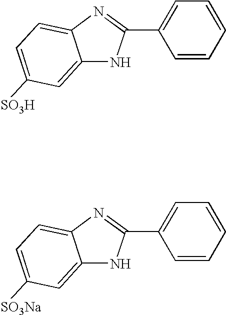
having the INCI name Phenylbenzimidazole Sulfonic Acid CCAS No.: 27503-81-7), which is obtainable from Merck, for example, under the trade name Eusolex 232 or from Haarmann & Reimer under Neo Heliopan Hydro.

A further advantageous sulfonated UV filter is 3,3′-(1,4-phenylenedimethylene)bis (7,7-dimethyl-2-oxobicyclo-[2.2.1 ]hept-1-ylmethane sulfonic acid, such as its sodium, potassium or its triethanolammonium salts, and the sulfonic acid itself: 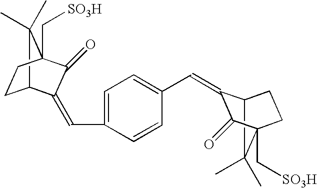
having the INCI name Terephthalidene Dicamphor Sulfonic Acid (CAS No.: 90457-82-2), which is obtainable, for example, from Chimex under the trade name Mexoryl SX.

Further advantageous water-soluble UV-B and/or broadband filter substances are, for example:
sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and their salts.

The total amount of one or more sulfonated UV filter substances in the finished cosmetic or dermatological preparations is advantageously chosen from the range 0.01% by weight to 20% by weight, preferably from 0.1 to 10% by weight, in each case based on the total weight of the preparations.

Advantageous UV filter substances within the meaning of the present invention are furthermore “broadband filters”, i.e. filter substances which absorb both UV-A and UV-B radiation.

Advantageous broadband filters or UV-B filter substances are, for example, bis-resorcinyltriazine derivatives having the following structure: 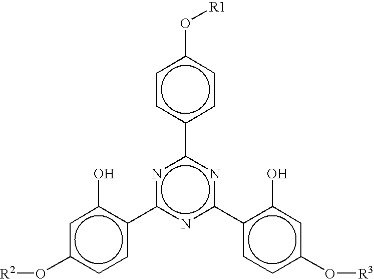
where R¹, R² and R³ independently of one another are chosen from the group consisting of the branched and unbranched alkyl groups having 1 to 10 carbon atoms or an individual hydrogen atom. 2,4-Bis{[4-(2-ethylhexyloxy)-2-hydroxy]phenyl}-6-(4-methoxyphenyl)-1,3,5-triazine (INCI: Bisethylhexyloxyphenol Methoxyphenyl Triazine), which is obtainable from CIBA Chemikalien GmbH under the trade name Tinosorb® S, are particularly preferred.

Particularly advantageous preparations within the meaning of the present invention, which are distinguished by a high or very high UV-A protection, contain, besides the filter substance(s) according to the invention, preferably further UV-A and/or broadband filters, in particular dibenzoylmethane derivatives [for example 4-(tert-butyl)-4′-methoxydibenzoylmethane], phenylene-1,4-bis(2-benzimidazyl)-3,3′-5,5′-tetrasulfonic acid and/or its salts, 2,2′-methylenebis(6-(2H-benzotriazol-2-yl)-4-(1,1,3,3-tetramethylbutyl)phenol), 1,4-di(2-oxo-10-sulfo-3-bornylidenemethyl)-benzene and/or its salts and/or 2,4-bis{[4-(2-ethylhexyloxy)-2-hydroxy]phenyl}-6-(4-methoxyphenyl)-1,3,5-triazine, in each case individually or in any desired combinations with one another.

Other UV filter substances which have the structural motif 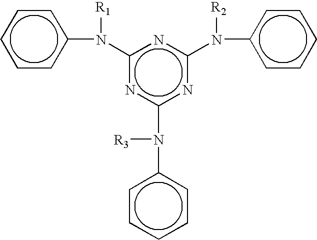
are also advantageous UV filter substances within the meaning of the present invention, for example the s-triazine derivatives described in European laid-open specification EP 570 838 A1, whose chemical structure is represented by the generic formula 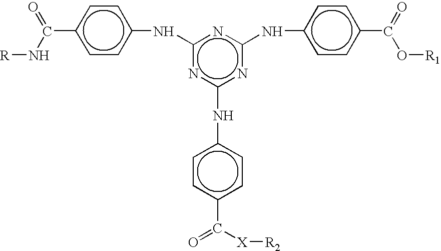
where
R is a branched or unbranched C₁-C₁₈-alkyl radical, a C₅-C₁₂-cycloalkyl radical, optionally substituted by one or more C₁-C₄-alkyl groups,
X is an oxygen atom or an NH group,
R₁ is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula 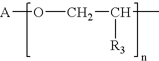
   in which
A is a branched or unbranched C₁-C₁₈-alkyl radical, a C₅-C₁₂-cycloalkyl or aryl radical, optionally substituted by one or more C₁-C₄-alkyl groups,
R₃ is a hydrogen atom or a methyl group,
n is a number from 1 to 10,
R₂ is a branched or unbranched C₁-C₁₈-alkyl radical, a C₅-C₁₂-cycloalkyl radical, optionally substituted by one or more C₁-C₄-alkyl groups, if X is the NH group, and
a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula 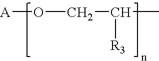
   in which
A is a branched or unbranched C₁-C₁₈-alkyl radical, a C₅-C₁₂-cycloalkyl or aryl radical, optionally substituted by one or more C₁-C₄-alkyl groups,
R₃ is a hydrogen atom or a methyl group,
n is a number from 1 to 10,
if X is an oxygen atom.

A particularly preferred UV filter substance within the meaning of the present invention is furthermore an unsymmetrically substituted s-triazine, whose chemical structure is represented by the formula 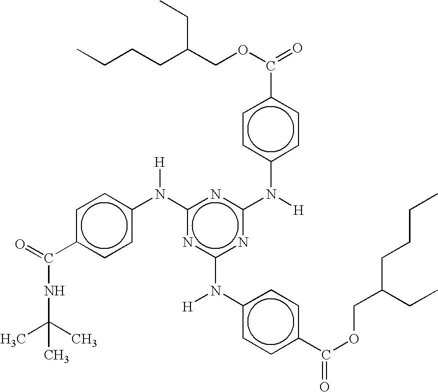
which is also designated as diethylhexylbutylamidotriazone (INCI: Diethylhexyl Butamidotriazone) below and is obtainable from Sigma 3V under the trade name UVASORB HEB.

Also advantageous within the meaning of the present invention is a symmetrically substituted s-triazine, 4,4′,4″-(1,3,5-triazine-2,4,6-triyltriimino)trisbenzoic acid tris(2-ethylhexyl ester), synonym: 2,4,6-tris[anilino(p-carbo-2′-ethyl-1′-hexyloxy)]-1,3,5-triazine (INCI: Ethylhexyl Triazone), which is marketed by BASF Aktiengesellschaft under the trade name UVINUL® T 150.

Also in European laid-open specification 775 698, bisresorcinyltriazine derivatives preferably to be employed are described, whose chemical structure is represented by the generic formula 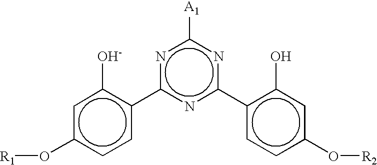
where R₁, R₂ and A₁ represent all sorts of organic radicals.

Furthermore advantageous within the meaning of the present invention are 2,4-bis-{[4-(3-sulfonato)-2-hydroxypropyloxy)-2-hydroxy]phenyl}-6-(4-methoxyphenyl)-1,3,5-triazine sodium salt, 2,4-bis-{[4-(3-(2-propyloxy)-2-hydroxypropyloxy)-2-hydroxy]phenyl}-6-(4-methoxyphenyl)-1,3,5-triazine, 2,4-bis{[4-(2-ethylhexyloxy)-2-hydroxy]phenyl}-6-[4-(2-methoxyethylcarboxyl)phenylamino]-1,3,5-triazine, 2,4-bis-{[4-(3-(2-propyloxy)-2-hydroxypropyloxy)-2-hydroxy]phenyl}-6-[4-(2-ethylcarboxyl)-phenylamino]-1,3,5-triazine, 2,4-bis-{[4-(2-ethylhexyloxy)-2-hydroxy]phenyl}-6-)1-methylpyrrol-2-yl)-1,3,5-triazine, 2,4-bis{[4-tris(trimethylsiloxysilylpropyloxy)-2-hydroxy]phenyl}-6-(4-methoxyphenyl)-1,3,5-triazine, 2,4-bis-{[4-(2″-methylpropenyl-oxy)-2-hydroxy]phenyl}-6-(4-methoxyphenyl)-1,3,5-triazine and 2,4-bis{[4-(1′,1′,1′,3′,5′,5′,5′-heptamethylsiloxy-2″-methylpropyloxy)-2-hydroxy]phenyl}-6-(4-methoxyphenyl)-1,3,5-triazine.

Additionally advantageous, within the meaning of the invention, are the benzotriazole derivatives. Benzotriazoles are distinguished by the following structural formula: 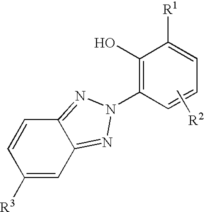
in which
R¹ and R² independently of one another can be linear or branched, saturated or unsaturated, substituted (e.g. substituted by a phenyl radical) or unsubstituted alkyl radicals having 1 to 18 carbon atoms and/or polymeric radicals which do not absorb UV rays themselves (such as, for example, silicone radicals, acrylate radicals and suchlike), and
R³ is chosen from the group consisting of H or an alkyl radical having 1 to 18 carbon atoms.

An advantageous benzotriazole within the meaning of the present invention is 2,2′-methylenebis(6-(2H-benzotriazol-2-yl)-4-( 1,1,3,3-tetramethylbutyl)phenol), a broadband filter, which is characterized by the chemical structural formula 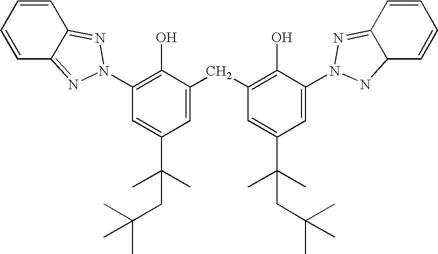
and is obtainable from CIBA Chemikalien GmbH under the trade name Tinosorb® M.

An advantageous benzotriazole within the meaning of the present invention is furthermore 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.:155633-54-8) having the INCI name Drometrizole Trisiloxane, which is characterized by the chemical structural formula

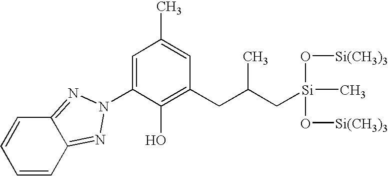

Further advantageous benzotriazoles within the meaning of the present invention are [2,4′-dihydroxy-3-(2H-benzotriazol-2-yl)-5-(1,1,3,3-tetramethylbutyl)-2′-n-octoxy-5′-benzoyl]diphenylmethane, 2,2′-methylenebis[6-(2H-benzotriazol-2-yl)-4-(methyl-phenol], 2,2′-methylenebis[6-(2H-benzotriazol-2-yl)-4-(1,1,3,3-tetramethylbutyl)-phenol], 2-(2′-hydroxy-5′-octylphenyl)benzotriazole, 2-(2′-hydroxy-3′,5′-di-t-amyl-phenyl)benzotriazole and 2-(2′-hydroxy-5′-methylphenyl)benzotriazole.

According to the invention, cosmetic or dermatological preparations contain 0.1 to 20% by weight, advantageously 0.5 to 15% by weight, very particularly preferably 0.5 to 10% by weight, of one or more benzotriazoles.

Liquid UV filter substances particularly advantageous at room temperature within the meaning of the present invention are homomenthyl salicylate, 2-ethylhexyl-2-cyano-3,3-diphenyl acrylate, 2-ethylhexyl 2-hydroxybenzoate and esters of cinnamic acid, preferably 4-methoxycinnamic acid (2-ethylhexyl) ester and 4-methoxycinnamic acid isopentyl ester.

Homomenthyl salicylate (INCI: Homosalate) is distinguished by the following structure:

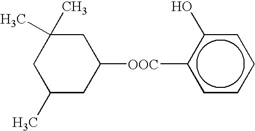

2-Ethylhexyl-2-cyano-3,3-diphenyl acrylate (INCI: Octocrylene) is obtainable from BASF under the name Uvinul® N 539 and is distinguished by the following structure:

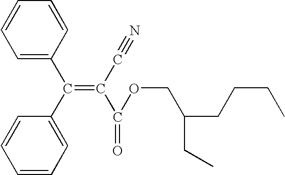

2-Ethylhexyl 2-hydroxybenzoate (2-ethylhexyl salicylate, octyl salicylate, INCI: Octyl Salicylate) is obtainable, for example, from Haarmann & Reimer under the trade name Neo Helipan OS and is distinguished by the following structure:

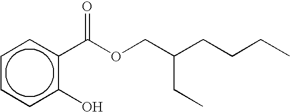

4-Methoxycinnamic acid (2-ethylhexyl) ester (2-ethylhexyl 4-methoxycinnamate, INCI: Octyl Methoxycinnamate) is obtainable from Hoffmann-la Roche under the trade name Parsol MCX and is distinguished by the following structure:

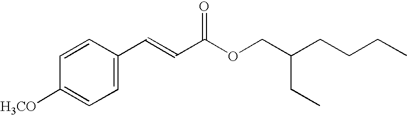

4-Methoxycinnamic acid isopentyl ester (isopentyl 4-methoxycinnamate, INCI: Iso-amyl p-Methoxycinnamate) is obtainable, for example, from Haarmann & Reimer under the trade name Neo Helipan E 1000 and is distinguished by the following structure:

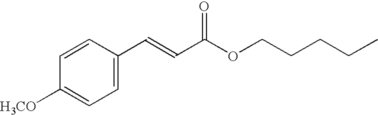

A further advantageous UV filter substance within the meaning of the present invention, which is liquid at room temperature (3-(4-(2,2-bisethoxycarbonylvinyl)-phenoxy)propenyl)methylsiloxane/dimethylsiloxane copolymer, which is obtainable, for example, from Hoffmann-Ia Roche under the trade name Parsol SLX.

The total amount of one or more UV filter substances which are liquid at room temperature in the finished cosmetic or dermatological preparations is advantageously chosen from the range 0.1% by weight to 30% by weight, preferably from 0.5 to 20% by weight, in each case based on the total weight of the preparations.

It can also be a considerable advantage to use polymer-pound or polymeric UV filter substances in preparations according to the present invention, in particular those such as are described in WO-A-92/20690.

The list of the UV filter substances mentioned which can be employed within the meaning of the present invention is not intended, of course, to be limiting.

Advantageously, the preparations according to the invention contain the substances which absorb UV radiation in the UV-A and/or UV-B range in a total amount of, for example, 0.1% by weight to 30% by weight, preferably 0.5 to 25% by weight, in particular 1.0 to 20% by weight, in each case based on the total weight of the preparations in order to make cosmetic preparations available which protect the hair or the skin from the entire range of ultraviolet radiation. They can also be used as sunscreens for the hair or the skin.

Furthermore, it can be advantageous to incorporate film-forming agents into the cosmetic or dermatological preparations according to the invention, for example in order to improve the water resistance of the preparations or to increase the UV protection power (UV-A and/or UV-B boosting). Both water-soluble and dispersible and also fat-soluble film-forming agents are suitable, in each case individually or in combination with one another.

Advantageous water-soluble or dispersible film-forming agents are, for example, polyurethanes (e.g. the Avalure® types from Goodrich), Dimethicone Copolyol Poly-acrylate (Silsoft Surface® from the Witco Organo Silicones group), PVP/VA (VA=vinyl acetate) copolymer (Luviscol VA 64 powder from BASF) etc.

Advantageous fat-soluble film-forming agents are, for example, the film-forming agents from the group consisting of the polymers based on polyvinylpyrrolidone (PVP)

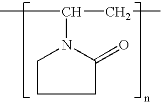

Copolymers of polyvinylpyrrolidone are particularly preferred, for example PVP hexadecene copolymer and PVP eicosene copolymer, which are obtainable under the trade names Antaron V216 and Antaron V220 from GAF Chemicals Cooperation, and Triacontyl PVP and suchlike.

Cleansing Agents

According to the invention, these emulsions can be employed as cosmetic and dermatological preparations and as cleansing agents.

Cosmetic preparations which are cosmetic cleansing preparations for the skin can be present in liquid or solid form. Besides active ingredient combinations according to the invention, they preferably contain at least one anionic, nonionic or amphoteric surface-active substance or mixtures thereof, if desired one or more electrolytes and excipients such as are customarily used therefor. The surface-active substance can be present in a concentration of between 1 and 94% by weight in the cleansing preparations, based on the total weight of the preparations.

Repellents—Insect-Repellent Agents

A further advantageous embodiment of the present invention consists in insect-repellent agents.

Advantageous active ingredients for repellents are low-melting or liquid amides, alcohols, esters and ethers having melting points of over 150° C., which evaporate only slowly at room temperature.

The following active ingredients have proven particularly advantageous individually in combination with one another or with others: 3-(N-n-butyl-N-acetylamino)propionic acid ethyl ester (trade name: Insect Repellent 3535 obtainable from Merck), N,N-di-ethyl-3-methylbenzamide (DEET), dimethyl phthalate, ethylhexanediol, caprylic acid diethylamide and natural plant oils such as citronella oil, eucalyptus oil, lavender oil and oil of cloves.

Self-Tanning Agents

A further advantageous embodiment of the present invention consists in self-tanning agents.

Advantageous active ingredients for self-tanning agents are natural or synthetic ketols or aldols. Dihydroxyacetone (DHA), glycerolaldehyde, erythrulose, melanin, alloxan, hydroxy-methylglyoxal, γ-dialdehyde, 6-aldo-D-fructose, ninhydrin and meso-tartaric acid di-aldehyde have proven advantageous.

Mixtures of the abovementioned active ingredients with one another or with muconic dialdehyde or/and naphthoquinones such as, for example, 5-hydroxy-1,4-naphthoquinone (juglone) have particularly advantageous.

Tissues

According to the invention, in combination with the highly liquid cosmetic and dermatological W/O impregnation emulsions, tissues are employed which consist of a nonwoven which is in particular water jet-consolidated and/or water jet-embossed (spunlaced material).

The macro embossing incorporated into the nonwoven can have any desired pattern. The choice to be made depends on on the one hand on the impregnation to be applied and on the other hand according to the field of use to which the future tissue is to be used.

Large cavities in the nonwoven surface and in the nonwoven facilitate the absorption of dirt and impurities if the skin is run over with the impregnated tissue. The cleansing action is increased by a large amount compared with the unimpregnated tissues.

Relative to the unembossed nonwoven, the thickness of the nonwoven with the high spots produced by embossing is advantageously approximately twice as high. In preferred embodiments, the embossed nonwoven is between 5% and 50%, very particularly preferably between 10% and 25%, thicker than the unembossed nonwoven.

The embossed nonwoven additionally has particular properties which make possible the use as a carrier material for emulsions or other preparations.

Thus the tensile strength is, in particular

| | | [N/50 mm] |
|---|---|---|
| in the dry state | machine direction | >60, preferably >80 |
| | transverse direction | >20, preferably >30 |
| in the impregnated state | machine direction | >4, preferably >60 |
| | transverse direction | >10, preferably >20 |
| The stretchability of | machine direction | 15% to 100%, preferably |
| the tissue is preferably | | 20% and 50% |
| in the dry state | | |
| | transverse direction | 40% to 120%, preferably |
| | | 50% and 85% |
| in the impregnated state | machine direction | 15% to 100%, preferably |
| | | 20% and 40% |
| | transverse direction | 40% to 120%, preferably |
| | | 50% and 85% |

It has turned out to be advantageous for the tissue if it has a weight of 35 to 120 g/m², preferably of 40 to 60 g/m², (measured at 20° C.±2° C. and with a humidity of the room air of 65%±5% for 24 hours).

The thickness of the nonwoven is preferably 0.4 mm to 1.5 mm, in particular 0.6 mm to 0.9 mm.

Finally, it is particularly advantageous for the tissue to have a “surface Tinting” of less than 4 mg/1000 mm², preferably less than 2 mg/1000 mm².

As starting materials for the nonwoven of the tissue, generally all organic and inorganic natural- and synthetic-based fibers can be used. Viscose, cotton, jute, hemp, sisal, silk, wool, polypropylene, polyester, polyethylene terephthalate (PET), aramid, nylon, polyvinyl derivatives, polyurethanes, polylactide, polyhydroxy-alkanoate, cellulose ester and/or polyethylene, and also mineral fibers such as glass fibers or carbon fibers can be mentioned. The present invention, however, is not restricted to the materials mentioned, but a multiplicity of further fibers can be employed for the formation of the nonwoven.

In a particularly advantageous embodiment of the nonwoven, the fibers consist of a mixture of 70% of viscose and 30% of PET.

Fibers of high-strength polymers such as polyamide, polyester and/or high-flex polyethylene are also particularly advantageous.

Moreover, the fibers can also be dyed in order to emphasize and/or to increase the visual attractiveness of the nonwoven. The fibers can additionally contain UV stabilizers and/or preservatives.

The fibers employed for the formation of the tissue preferably have a water absorption rate of more than 60 mm/[10 min] (measured using the EDANA test 10.1-72), in particular more than 80 mm/[10 min].

The fibers employed for the formation of the tissue preferably then have a water absorption power of more than 5 g/g (measured using the EDANA test 10.1-72), in particular more than 8 g/g.

### DETAILED DESCRIPTION OF THE INVENTION

The following examples are intended to illustrate the impregnation solutions according to the invention without restricting them. The numerical values in the examples denote percentages by weight, based on the total weight of the respective preparations.

### EXAMPLES

The following examples are intended to illustrate the present invention without restricting it. The numerical values in the examples denote percentages by weight, based on the total weight of the respective preparations.

A. Impregnation medium: W/O sunscreen emulsions

| 1. | |
|---|---|
| Cetyl dimethicone copolyol | 2 |
| Polyglyceryl-2 dipolyhydroxy-stearate | 2 |
| Polysorbate-65 | 1 |
| PEG-100 stearate | 0.5 |
| Cetyl phosphate | 1 |
| Cyclomethicone | 10 |
| Caprylyl methicone | 5 |
| Tinosorb ® S | 2 |
| Ethylhexyl triazone | 4 |
| Octocrylene | 5 |
| Ethylhexyl salicylate | 5 |
| Phenylbenzimidazole sulfonate | 4 |
| Titanium dioxide T 805 ® | 3 |
| Zinc oxide neutral | 1 |
| C₁₂₋₁₅ alkyl benzoate | 2 |
| Butylene glycol dicaprylate/dicaprate | 5 |
| Dicaprylyl carbonate | 3 |
| Dihexyl carbonate | 5 |
| Shea butter | 0.75 |
| PVP hexadecene copolymer | 0.5 |
| Silsoft Surface ® | 1.0 |
| Glycerol | 10 |
| Xanthan gum | 0.1 |
| Vitamin E acetate | 1 |
| EDTA | 0.01 |
| Magnesium sulfate | 0.3 |
| DMDM hydantoin | 0.01 |
| Ethanol | 4 |
| Dye | q.s. |
| Perfume | q.s. |
| Water | to 100 |
| 2. | |
| Lauryl methicone copolyol | 3 |
| Polyglyceryl-3 diisostearate | 2 |
| Polysorbate-20 | 2 |
| Cetearyl sulfate | 0.7 |
| Dimethicone | 2 |
| Phenyl trimethicone | 5 |
| Tinosorb ® S | 3 |
| 4-Methylbenzylidene camphor | 4 |
| Ethylhexyl methoxycinnamate | 10 |
| Homosalate | 7 |
| Diethylhexyl butamidotriazone | 2 |
| Dimethico-diethylbenzal-malonate | 3 |
| MT-100 Z ® | 2 |
| Z-Cote HP1 | 3 |
| Dicaprylyl ether | 6 |
| Butylene glycol dicaprylate/dicaprate | 2 |
| Mineral oil | 7 |
| PVP hexadecene copolymer | 1.0 |
| Glycerol | 7.5 |
| Vitamin E acetate | 0.5 |
| Magnesium sulfate | 0.7 |
| Konkaben LMB ® | 0.12 |
| Methylparaben | 0.3 |
| Phenoxyethanol | 0.5 |
| Dye | q.s. |
| Perfume | q.s. |
| Water | to 100 |
| 3. | |
| Cetyl dimethicone copolyol | 1.5 |
| Lauryl methicone copolyol | 0.7 |
| Polyglyceryl-2 dipolyhydroxy-stearate | 1.0 |
| Polysorbate-65 | 1 |
| PEG-100 stearate | 1 |
| Cyclomethicone | 15 |
| Neo Heliopan AP ® | 2 |
| Butyl methoxydibenzoyl-methane | 1 |
| Ethylhexyl triazone | 2 |
| 4-methylbenzylidene camphor | 4 |
| Ethylhexyl salicylate | 10 |
| Phenylbenzimidazole sulfonate | 1.5 |
| C₁₂₋₁₅ alkyl benzoate | 5 |
| Dicaprylyl carbonate | 4 |
| Dihexyl carbonate | 6 |
| Shea butter | 3 |
| Silsoft Surface ® | 0.50 |
| Glycerol | 5 |
| Butylene glycol | 5 |
| Xanthan gum | 0.3 |
| Sodium chloride | 1.2 |
| Glycine soya | 1.5 |
| Ethanol | 5 |
| Dye | q.s. |
| Perfume | q.s. |
| Water | to 100 |
| 4. | |
| Cetyl dimethicone copolyol | 2.5 |
| Isostearyl diglyceryl succinate | 1.5 |
| Cetyl phosphate | 1.2 |
| Dimethicone | 3 |
| Phenyl trimethicone | 10 |
| Tinosorb ® S | 1 |
| Tinosorb M ® | 2 |
| Ethylhexyl triazone | 1.5 |
| Ethylhexyl methoxycinnamate | 5 |
| Homosalate | 7 |
| Dimethicone diethylbenzal-malonate | 0.5 |
| Octyl cocoate | 4 |
| Mineral oil | 5 |
| Vitamin E acetate | 0.3 |
| α-Glucosylrutin | 0.25 |
| EDTA | 0.2 |
| Magnesium sulfate | 1 |
| Sodium chloride | 0.1 |
| Glycine soya | 1 |
| Ethanol | 3 |
| Dye | q.s. |
| Perfume | q.s. |
| Water | to 100 |
| 5. | |
| Cetyl dimethicone copolyol | 1.5 |
| Polyglyceryl-2 dipolyhydroxy-stearate | 2 |
| Polysorbate-20 | 1 |
| Cetearyl sulfate | 0.5 |
| Cyclomethicone | 3 |
| Neo Heliopan AP ® | 0.5 |
| Butyl methoxydibenzoyl-methane | 1.5 |
| Tinosorb M ® | 2 |
| Ethylhexyl salicylate | 8 |
| Dimethico-diethylbenzal-malonate | 1 |
| Z-Cote HP1 | 1.5 |
| C₁₂₋₁₅ alkyl benzoate | 7.5 |
| Dicaprylyl carbonate | 10 |
| Glycerol | 7.5 |
| Vitamin E acetate | 1.5 |
| Sodium chloride | 0.6 |
| DMDM hydantoin | 0.02 |
| Methylparaben | 0.4 |
| Dye | q.s. |
| Perfume | q.s. |
| Water | to 100 |
| 6. | |
| Cetyl dimethicone copolyol | 3 |
| Polyglyceryl-2 dipolyhydroxy-stearate | 1 |
| Isostearyl diglyceryl succinate | 0.3 |
| Polysorbate-65 | 1.5 |
| Cetyl phosphate | 0.7 |
| Cetearyl sulfate | 1 |
| Dimethicone | 2 |
| Cyclomethicone | 15 |
| Tinosorb ® S | 4 |
| Ethylhexyl methoxycinnamate | 10 |
| Octocrylene | 7.5 |
| Ethylhexyl salicylate | 6.5 |
| Phenylbenzimidazole sulfonate | 4 |
| MT-100 Z ® | 0.5 |
| Zinc oxide neutral | 4 |
| Dicaprylyl carbonate | 4 |
| Dihexyl carbonate | 6 |
| Mineral oil | 6 |
| PVP hexadecene copolymer | 0.4 |
| Butylene glycol | 7 |
| α-Glucosylrutin | 0.15 |
| EDTA | 0.15 |
| Magnesium sulfate | 1 |
| Konkaben LMB ® | 0.1 |
| Phenoxyethanol | 1 |
| Repellent 3535 ® | 10.0 |
| Ethanol | 1 |
| Dye | q.s. |
| Perfume | q.s. |
| Water | to 100 |
| 7. | |
| Cetyl dimethicone copolyol | 1 |
| Lauryl methicone copolyol | 2.5 |
| Isostearyl diglyceryl succinate | 1 |
| Polysorbate-20 | 1 |
| Caprylyl methicone | 5 |
| Neo Heliopan AP ® | 1 |
| Tinosorb ® S | 1 |
| Butyl methoxydibenzoyl-methane | 1 |
| Tinosorb M ® | 4 |
| Ethylhexyl triazone | 3 |
| Ethylhexyl methoxycinnamate | 10 |
| Titanium dioxide T 805 ® | 2.5 |
| Z-Cote HP1 | 7 |
| C₁₂₋₁₅ alkyl benzoate | 5 |
| Butylene glycol dicaprylate/ | 3 |
| dicaprate | |
| Octyl cocoate | 7.5 |
| Shea butter | 3 |
| Silsoft Surface ® | 0.75 |
| Glycerol | 15 |
| Xanthan gum | 0.5 |
| Vitamin E acetate | 1.0 |
| Magnesium sulfate | 1 |
| Konkaben LMB ® | 0.2 |
| Methylparaben | 0.3 |
| Dye | q.s. |
| Perfume | q.s. |
| Water | to 100 |
| 8. | |
| Cetyl dimethicone copolyol | 2.5 |
| Lauryl methicone copolyol | 0.7 |
| Polyglyceryl-2 dipolyhydroxy-stearate | 1.0 |
| Polysorbate-65 | 1 |
| PEG-100 stearate | 1 |
| Cyclomethicone | 20 |
| Tinosorb ® S | 3 |
| Butyl methoxydibenzoyl-methane | 1.5 |
| Tinosorb M ® | 1 |
| 4-Methylbenzylidene camphor | 1 |
| Octocrylene | 4 |
| Ethylhexyl salicylate | 8 |
| Homosalate | 2 |
| Diethylhexyl butamidotriazone | 2 |
| Phenylbenzimidazole sulfonate | 2 |
| Titanium dioxide T 805 ® | 5 |
| PVP hexadecene copolymer | 0.7 |
| Butylene glycol | 7.5 |
| α-Glucosylrutin | 0.5 |
| Magnesium sulfate | 0.7 |
| DMDM hydantoin | 0.01 |
| Glycine soya | 0.5 |
| Dye | q.s. |
| Perfume | q.s. |
| Water | to 100 |
| 9. | |
| Cetyl dimethicone copolyol | 3 |
| Polyglyceryl-2 dipolyhydroxy-stearate | 2 |
| Polysorbate-65 | 0.5 |
| PEG-100 stearate | 0.5 |
| Cetyl phosphate | 1 |
| Dimethicone | 5 |
| Cyclomethicone | 7 |
| Caprylyl methicone | 6 |
| Neo Heliopan AP ® | 2.5 |
| Butyl methoxydibenzoyl-methane | 2 |
| Ethylhexyl triazone | 2 |
| Octocrylene | 2.5 |
| Dimethico-diethylbenzal-malonate | 2 |
| Dicaprylyl carbonate | 5 |
| Dihexyl carbonate | 5 |
| Mineral oil | 15 |
| Shea butter | 2 |
| Glycerol | 4 |
| Butylene glycol | 5 |
| Vitamin E acetate | 0.75 |
| Sodium chloride | 0.75 |
| Phenoxyethanol | 1 |
| Glycine soya | 1 |
| Dye | q.s. |
| Perfume | q.s. |
| Water | to 100 |
| 10. | |
| Cetyl dimethicone copolyol | 1.5 |
| Polyglyceryl-3 diisostearate | 2 |
| Polysorbate-65 | 2 |
| Cetearyl sulfate | 0.75 |
| Dimethicone | 5 |
| Cyclomethicone | 5 |
| Phenyl trimethicone | 2 |
| Neo Heliopan AP ® | 1 |
| Tinosorb ® S | 2 |
| Ethylhexyl triazone | 3 |
| Ethylhexyl methoxycinnamate | 5 |
| Dicaprylyl ether | 8 |
| Butylene glycol dicaprylate/dicaprate | 8 |
| Dicaprylyl carbonate | 3 |
| Glycerol | 6 |
| Butylene glycol | 10 |
| Sodium chloride | 1 |
| Methylparaben | 0.2 |
| Ethanol | 7 |
| Dye | q.s. |
| Perfume | q.s. |
| Water | to 100 |

B. Impregnation medium: caring W/O emulsions

| | 1 | 2 |
|---|---|---|
| Cetyl dimethicone copolyol | 2 | |
| Laurylmethicone copolyol | | 3 |
| Polyglyceryl-2 dipolyhydroxystearate | 1.5 | |
| Polyglyceryl-3 diisostearate | | 2 |
| Polysorbate-65 | 1 | |
| Polysorbate-20 | | 2 |
| PEG-100 stearate | 0.5 | |
| Trilaureth-4 phosphate | 1.5 | |
| Cetearyl sulfate | | 0.7 |
| Dimethicone | 5 | |
| Cyclomethicone | 5 | 15 |
| Phenyl trimethicone | 2 | |
| Caprylyl methicone | | 1 |
| C₁₂₋₁₅ alkyl benzoate | | 4 |
| Dicaprylyl ether | 10 | |
| Octyldodecanol | | 3 |
| Dicaprylyl carbonate | 10 | |
| Octyl cocoate | | 2 |
| Caprylic/capric triglyceride | | 2 |
| Shea butter | | 0.5 |
| Glycerol | 10 | 7 |
| Butylene glycol | | 10 |
| Vitamin E acetate | 1 | 0.5 |
| α-Glycosylrutin | | 0.15 |
| Magnesium sulfate | 0.7 | 1.4 |
| DMDM hydantoin | 0.01 | |
| Konkaben LMB ® | | 0.1 |
| Phenoxyethanol | 1 | 0.4 |
| Dihydroxyacetone | | 5 |
| Dye | q.s. | q.s. |
| Perfume | q.s. | q.s. |
| Water | to 100 | to 100 |

| | 3 | 4 |
|---|---|---|
| Cetyl dimethicone copolyol | | 2.5 |
| Laurylmethicone copolyol | 1.5 | |
| Polyglyceryl-2 dipolyhydroxy- | 2 | |
| stearate | | |
| Polyglyceryl-3 diisostearate | | |
| Isostearyl diglyceryl succinate | 0.7 | 1.5 |
| PEG-100 stearate | 1 | |
| Trilaureth-4 phosphate | | 1.2 |
| Dimethicone | | 1 |
| Phenyl trimethicone | | 7 |
| Caprylyl methicone | 10 | |
| C₁₂₋₁₅ alkyl benzoate | 8 | |
| Dicaprylyl carbonate | 4 | |
| Caprylic/capric triglyceride | | 5 |
| Isononyl octanoate | 10 | 5 |
| Dihexyl carbonate | | |
| Mineral oil | 10 | |
| Shea butter | | 1 |
| Glycerol | 15 | |
| Butylene glycol | | 5 |
| Xanthan gum | 0.2 | |
| Vitamin E acetate | 1 | |
| α-Glycosylrutin | | 0.3 |
| Coenzyme Q10 | 0.7 | |
| Sodium chloride | 1 | 1.5 |
| DMDM hydantoin | | |
| Konkaben LMB ® | 0.15 | 0.2 |
| Methylparaben | | 0.3 |
| Ethanol | | 2 |
| Dye | q.s. | q.s. |
| Perfume | q.s. | q.s. |
| Water | to 100 | to 100 |

| | 5 | 6 |
|---|---|---|
| Cetyl dimethicone copolyol | 1.5 | 3 |
| Polyglyceryl-2 dipolyhydroxy-stearate | 1.5 | 1 |
| Isostearyl diglyceryl succinate | | 0.3 |
| Polysorbate-65 | | 1.5 |
| Polysorbate-20 | 0.7 | |
| Cetearyl sulfate | | 1 |
| Dimethicone | 4 | |
| Cyclomethicone | 20 | |
| Caprylyl methicone | | 8 |
| C₁₂₋₁₅ alkyl benzoate | | 5 |
| Dicaprylyl ether | | 5 |
| Dicaprylyl carbonate | 10 | 15 |
| Isononyl octanoate | | 2 |
| Dihexyl carbonate | | 6 |
| Mineral oil | | 5 |
| Shea butter | 2 | |
| Glycerol | 5 | 7.5 |
| Butylene glycol | 5 | |
| Xanthan gum | | 0.5 |
| Vitamin E acetate | 0.75 | 2 |
| α-Glycosylrutin | | 0.2 |
| Coenzyme Q10 | | |
| Magnesium sulfate | 0.2 | 1 |
| Sodium chloride | 0.5 | |
| Phenoxyethanol | | 0.3 |
| Glycine soja | 1 | 0.7 |
| Ethanol | 5 | |
| Dihydroxyacetone | 7.5 | |
| Dye | q.s. | q.s. |
| Perfume | q.s. | q.s. |
| Water | to 100 | to 100 |
| | 7 | 8 |
| Cetyl dimethicone copolyol | 1 | 1.5 |
| Lauryl methicone copolyol | 2.5 | 0.7 |
| Polyglyceryl-2 dipolyhydroxy-stearate | | 1.0 |
| Isostearyl diglyceryl succinate | 1 | |
| Polysorbate-65 | | 1 |
| Polysorbate-20 | 1 | |
| PEG-100 stearate | | 1 |
| Dimethicone | 7 | 2 |
| Cyclomethicone | | 20 |
| Phenyl trimethicone | 15 | |
| Dicaprylyl ether | | 10 |
| Octyldodecanol | 5 | |
| Dicaprylyl carbonate | | 7.5 |
| Octyl cocoate | 7 | |
| Caprylic/capric triglyceride | | 2 |
| Glycerol | 10 | |
| Butylene glycol | | 10 |
| Vitamin E acetate | 1.5 | 0.5 |
| α-Glycosilrutin | | |
| Coenzyme Q10 | 0.02 | |
| Magnesium sulfate | 0.5 | 0.3 |
| DMDM hydantoin | 0.01 | |
| Methylparaben | 0.2 | |
| Glycine soya | | 1.5 |
| Ethanol | | 3 |
| Dye | q.s. | q.s. |
| Perfume | q.s. | q.s. |
| Water | to 100 | to 100 |

| | 9 | 10 |
|---|---|---|
| Cetyl dimethicone copolyol | 3 | 1.5 |
| Polyglyceryl-3 diisostearate | 1 | 2 |
| Polysorbate-65 | | 2 |
| Trilaureth-4 phosphate | 1 | |
| Cetearyl sulfate | | 0.75 |
| Cyclomethicone | 15 | |
| Phenyl trimethicone | | 4 |
| Caprylyl methicone | 5 | |
| C₁₂₋₁₅ alkyl benzoate | 9 | |
| Dicaprylyl ether | 5 | |
| Octyldodecanol | | |
| Dicaprylyl carbonate | 10 | |
| Octyl cocoate | | 15 |
| Caprylic/capric triglyceride | | 10 |
| Isononyl octanoate | | 4 |
| Dihexyl carbonate | | 5 |
| Mineral oil | | 15 |
| Shea butter | 4 | |
| Glycerol | 7.5 | 5 |
| Xanthan gum | 0.1 | |
| Vitamin E acetate | 0.3 | 0.2 |
| Magnesium sulfate | | 0.7 |
| Sodium chloride | 0.5 | |
| Konkaben LMB ® | 0.18 | |
| Methylparaben | 0.1 | |
| Phenoxyethanol | 1 | 1 |
| Glycine soya | | 0.5 |
| Dye | q.s. | q.s. |
| Perfume | q.s. | q.s. |
| Water | to 100 | to 100 |

1. A cosmetic or dermatological tissue comprising a water-insoluble nonwoven which is at least one of impregnated and moistened with a cosmetic or dermatological W/O emulsion, wherein the emulsion comprises (a) a water phase, (b) at least one oil phase which comprises one or more oils, one or more lipids and combinations thereof, and (c) an emulsifier system of
(A) at least one O/W emulsifier having an HLB value of >10; and at least one of
(B) at least one silicone emulsifier (W/S) having an HLB value of ≦8, and
(C) at least one W/O emulsifier having an HLB value of <7,
the emulsion having a viscosity of less than 2,000 mPa·s and a silicone oil content of not more 25% by weight.

2. The tissue of claim 1, wherein the weight ratio of the nonwoven and the W/O emulsion is from 5:1 to 1:5.

3. The tissue of claim 2, wherein the nonwoven comprises a structured nonwoven.

4. The tissue of claim 2, wherein the nonwoven comprises an unstructured nonwoven.

5. The tissue of claim 1, wherein the nonwoven comprises at least one of a jet-consolidated nonwoven and a water jet-embossed nonwoven.

6. The tissue of claim 2, wherein the nonwoven has a thickness of from 0.4 mm to 1.5 mm.

7. The tissue of claim 2, wherein the nonwoven has an area weight of from 35 to 120 g/m².

8. The tissue of claim 1, wherein the nonwoven has a thickness of from 0.6 mm to 0.9 mm and an area weight of from 40 to 60 g/m².

9. The tissue of claim 2, wherein the nonwoven comprises fibers of a mixture of 70% by weight of viscose and 30% by weight of polyethylene terephthalate.

10. The tissue of claim 8, wherein the nonwoven comprises fibers having at least one of a water absorption rate of more than 60 mm/10 min and a water absorption capacity of more than 5 g/g.

11. The tissue of claim 8, wherein the nonwoven comprises fibers having at least one of a water absorption rate of more than 80 mm/10 min and a water absorption capacity of more than 8 g/g.

12. The tissue of claim 1, wherein the at least one silicone emulsifier B comprises at least one of an alkylmethicone copolyol and an alkyl dimethicone copolyol.

13. The tissue of claim 12, wherein the at least one silicone emulsifier B comprises an emulsifier of the formula 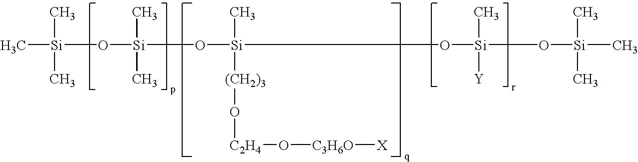
in which X and Y independently represent H, a branched or unbranched alkyl group, an acyl group and an alkoxy group having 1-24 carbon atoms, p is a number of from 0-200, q is a number of from 1-40, and r is a number of from 1-100.

14. The tissue of claim 1, wherein the at least one W/O emulsifier C comprises at least one polyglycerol emulsifier.

15. The tissue of claim 1, wherein the at least one O/W emulsifier A comprises at least one of an ethoxylated polysorbate, an ethoxylated stearate, a phosphate emulsifier and a sulfate emulsifier.

16. The tissue of claim 2, wherein the emulsion comprises a total concentration of A, B and C of from 0.1% to 15% by weight.

17. The tissue of claim 1, wherein the emulsion comprises a total concentration of A, B and C of from 0.5% to 10% by weight.

18. The tissue of claim 17, wherein the emulsion comprises a total concentration of A, B and C of at least 2% by weight.

19. The tissue of claim 16, wherein the weight ratio A:B:C is a:b:c and a, b and c are rational numbers of from 1 to 5.

20. The tissue of claim 19, wherein a, b and c are rational numbers of from 1 to 3.

21. The tissue of claim 1, wherein the emulsion comprises from 0.5% to 5.0% by weight of the at least one silicone emulsifier B.

22. The tissue of claim 1, wherein the emulsion comprises at least 2% by weight of one or more silicone oils which comprise at least one of a cyclic silicone, a linear silicone and a derivative thereof.

23. The tissue of claim 1, wherein the emulsion comprises from 2% to 25% by weight of at least one silicone oil.

24. The tissue of claim 16, wherein the emulsion comprises from 5% to 20% by weight of at least one silicone oil.

25. The tissue of claim 17, wherein the emulsion comprises from 10% to 20% by weight of at least one silicone oil.

26. The tissue of claim 1, wherein the at least one oil phase comprises at least one of a polar oil, a carboxylic acid ester, a dialkyl ether and a dialkyl carbonate.

27. The tissue of claim 26, wherein the at least one oil phase comprises a C₁₂₋₁₅ alkyl benzoate.

28. The tissue of claim 1, wherein the emulsion comprises from 1% to 90% by weight of the at least one oil phase.

29. The tissue of claim 17, wherein the emulsion comprises from 2.5% to 80% by weight of the at least one oil phase.

30. The tissue of claim 16, wherein the emulsion comprises from 5% to 70% by weight of the at least one oil phase.

31. The tissue of claim 1, wherein the emulsion further comprises at least one light protection filter selected from oil-soluble and water-soluble light protection filters.

32. The tissue of claim 31, wherein the at least one light protection filter comprises one or more UV filters.

33. The tissue of claim 32, wherein the one or more UV filters comprise at least one of a triazine, a sulfonated UV filter, a UV filter which is liquid at room temperature, an inorganic pigment and a benzotriazole.

34. The tissue of claim 32, wherein the one or more UV filters comprise at least one of 2,4-bis{[4-(2-ethylhexyloxy)2-hydroxy]phenyl}-6-(4-methoxyphenyl)-1,3,5-triazine, dioctylbutylamidotriazine, 4,4′,4″-(1,3,5-triazine-2,4,6-triyltriimino)trisbenzoic acid tris(2-ethylhexyl ester), phenylene-1,4-bis(2-benzimidazyl)-3,3′,5,5′-tetrasulfonic acid bis sodium salt, 2-phenylbenzimidazole-5-sulfonic acid, terephthalidene dicamphorsulfonic acid, 4-methoxycinnamic acid (2-ethylhexyl)ester, 2-ethylhexyl-2-cyano-3,3-diphenyl acrylate, 2-ethylhexyl 2-hydroxy-benzoate, homomenthyl salicylate, TiO₂, ZnO, 2,2′-methylenebis(6-(2H-benzotriazol-2-yl)-4-(1,1,3,3-tetramethylbutyl)phenol, and 2-(2H-benzotriazol-2-yl)-4-methyl-6-[2-methyl-3-[1,3,3,3-tetramethyl[(trimethylsilyl)oxy]disiloxanyl]propyl]-phenol.

35. The tissue of claim 1, wherein the emulsion further comprises at least one of an additive and an active ingredient.

36. The tissue of claim 35, wherein the emulsion further comprises at least one of a repellent, a self-tanning agent and a pigment.

37. The tissue of claim 1, wherein the emulsion further comprises at least one of vitamin E and a derivative thereof.

38. The tissue of claim 1, wherein the emulsion further comprises at least one of α-glycosylrutin and a derivative thereof.

39. The tissue of claim 1, wherein the emulsion further comprises at least one component selected from moisturizers, waxes, surfactants, preservatives, antioxidants, dyes, plant extracts, deodorants, antiperspirants, dermatologically active ingredients, and perfumes.

40. The tissue of claim 1, wherein the emulsion has a high water resistance.

41. A cosmetic or dermatological tissue comprising a water-insoluble nonwoven which is one of impregnated and moistened with a cosmetic or dermatological W/O emulsion, wherein the emulsion comprises (a) a water phase, (b) from 5% to 70% by weight of at least one oil phase which comprises one or more oils, one or more lipids and combinations thereof, and (c) an emulsifier system of
(A) at least one O/W emulsifier having an HLB value of >10; and at least one of
(B) at least one silicone emulsifier (W/S) having an HLB value of ≦8, and
(C) at least one W/O emulsifier having an HLB value of <7,
wherein A, B and C are present in a total concentration of from 0.5% to 10% by weight, and wherein the emulsion has a viscosity of less than 1,500 mPa·s and comprises up to 20% by weight of at least one silicone oil.

42. The tissue of claim 41, wherein the at least one silicone emulsifier B comprises at least one of an alkylmethicone copolyol and an alkyl dimethicone copolyol.

43. The tissue of claim 41, wherein the at least one W/O emulsifier C comprises at least one polyglycerol emulsifier.

44. The tissue of claim 41, wherein the at least one O/W emulsifier A comprises at least one of an ethoxylated polysorbate, an ethoxylated stearate, a phosphate emulsifier and a sulfate emulsifier.

45. The tissue of claim 41, wherein the at least one oil phase comprises at least one of a polar oil, a carboxylic acid ester, a dialkyl ether and a dialkyl carbonate.

46. A skin care product which comprises the tissue of claim 1.

47. An insect repellent which comprises the tissue of claim 1.

48. A self-tanning product which comprises the tissue of claim 1.

49. A sunscreen product which comprises the tissue of claim 1.

50. A product for the treatment or prophylaxis of light-related skin ageing which comprises the tissue of claim 1.

51. A skin moisturizing product which comprises the tissue of claim 1.

52. A baby care product which comprises the tissue of claim 1.

53. A skin cleansing product which comprises the tissue of claim 1.

54. A cosmetic or dermatological W/O emulsion for impregnating or moistening a water-insoluble nonwoven, wherein the emulsion comprises (a) a water phase, (b) at least one oil phase which comprises one or more oils, one or more lipids and combinations thereof, and (c) an emulsifier system of
(A) at least one O/W emulsifier having an HLB value of >10; and at least one of
(B) at least one silicone emulsifier (W/S) having an HLB value of ≦8, and
(C) at least one W/O emulsifier having an HLB value of <7,
the emulsion having a viscosity of less than 2,000 mPa·s, and a silicone oil content of not more 25% by weight.

55. The emulsion of claim 54, wherein the at least one silicone emulsifier B comprises at least one of an alkylmethicone copolyol and an alkyl dimethicone copolyol.

56. The emulsion of claim 54, wherein the at least one silicone emulsifier B comprises an emulsifier of the formula 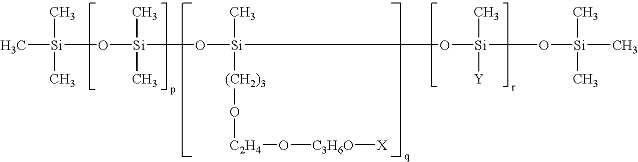
in which X and Y independently represent H, a branched or unbranched alkyl group, an acyl group and an alkoxy group having 1-24 carbon atoms, p is a number of from 0-200, q is a number of from 1-40, and r is a number of from 1-100.

57. The emulsion of claim 54, wherein the at least one W/O emulsifier C comprises at least one polyglycerol emulsifier.

58. The emulsion of claim 54, wherein the at least one O/W emulsifier A comprises at least one of an ethoxylated polysorbate, an ethoxylated stearate, a phosphate emulsifier and a sulfate emulsifier.

59. The emulsion of claim 54, wherein the emulsion comprises a total concentration of A, B and C of from 0.5% to 10% by weight.

60. The emulsion of claim 59, wherein the weight ratio A:B:C is a:b:c and a, b and c are rational numbers of from 1 to 3.

61. The emulsion of claim 54, wherein the emulsion comprises from 0.5% to 5.0 % by weight of the at least one silicone emulsifier B.

62. The emulsion of claim 59, wherein the emulsion comprises from 5% to 20% by weight of at least one silicone oil.

63. The emulsion of claim 54, wherein the at least one oil phase comprises at least one of a polar oil, a carboxylic acid ester, a dialkyl ether and a dialkyl carbonate.

64. The emulsion of claim 62, wherein the emulsion comprises from 5% to 70% by weight of the at least one oil phase.

65. The emulsion of claim 54, wherein the emulsion further comprises at least one of vitamin E and a derivative thereof and α-glycosylrutin and a derivative thereof.

66. The emulsion of claim 54, wherein the emulsion has a viscosity of less than 1,500 mPa.s and comprises from 5% to 70% by weight of the at least one oil phase, a total concentration of A, B and C of from 0.5% to 10% by weight, and up to 20% by weight of the at least one silicone oil.

67. A process for manufacturing the tissue of claim 1, wherein the process comprises providing a water-insoluble nonwoven and at least one of impregnating and moistening the nonwoven with a cosmetic or dermatological W/O emulsion which comprises (a) a water phase, (b) at least one oil phase which comprises one or more oils, one or more lipids and combinations thereof, and (c) an emulsifier system of
(A) at least one O/W emulsifier having an HLB value of >10; and at least one of
(B) at least one silicone emulsifier (W/S) having an HLB value of ≦8, and
(C) at least one W/O emulsifier having an HLB value of <7,
the emulsion having a viscosity of less than 2,000 mPa·s and a silicone oil content of not more 25% by weight.
